(19) United States
(12) Patent Application Publication
(10) Pub. No.: US 2024/0336938 A1
(43) Pub. Date: Oct. 10, 2024

(54) COMPOSITIONS AND METHODS FOR EFFECTIVE DELIVERY OF POLYNUCLEOTIDES TO CELLS

(71) Applicant: WUHAN UNIVERSITY, Wuhan, Hubei (CN)

(72) Inventors: Ying ZHANG, Wuhan, Hubei (CN); Hao YIN, Wuhan, Hubei (CN); Chuanping ZHANG, Wuhan, Hubei (CN); Jing AN, Wuhan, Hubei (CN)

(21) Appl. No.: 18/579,305

(22) PCT Filed: Jul. 19, 2022
(86) PCT No.: PCT/CN2022/106565

§ 371 Date: Jan. 12, 2024

(30) Foreign Application Priority Data
Jul. 19, 2021 (WO) PCT/CN2021/107047

(51) Int. Cl. C12N 15/87; C12N 5/00; C12N 5/071; C12N 13/00

(52) U.S. Cl. CPC C12N 15/87; C12N 5/0018; C12N 5/0602; C12N 13/00; C12N 2500/12; C12N 2500/32; C12N 2500/34; C12N 2500/35; C12N 2501/01; C12N 2501/999

(57) ABSTRACT

Provided are compositions and methods for effective delivery of polynucleotides into the nuclei of cells through electroporation. A new buffer system is provided that facilitates the delivery with greatly reduced cell toxicity. The buffer may include succinate, mannitol, a sugar, glutamine or an analog, and an antioxidant. Also provided are methods that are particularly suitable for delivering an RNA or protein into a cell by electrophoresis in a solution having an osmotic pressure greater than 310 mOsmol/kg.

### BACKGROUND

Non-viral genome editing for targeted insertion to reprogram T cells holds great therapeutic potentials. However, electroporation-mediated DNA delivery causes severe cellular toxicity, limiting its broad applications.

Non-viral reprogramming of primary human T cells provides a safe, fast and virus-free generation of CAR-T cells. Unlike lentivirus-mediated random integration or site-specific integration of CAR construct using AAV as templates, non-viral reprogramming of CAR-T cells circumvents the need of lentivirus or AAV preparation for donor DNA delivery and can be quickly adopted for manufactory process development. However, the biggest hurdle that holds back the non-viral delivery is the cellular toxicity after the electroporation of DNA donor and the relative low delivery efficiency into human T cells.

Improved methods for effective delivery of polynucleotides into cells, in particular into nuclei in the cells, are needed.

### SUMMARY

The present disclosure, in certain embodiments, provides compositions and methods for effective delivery of polynucleotides into cells, in particular into the nuclei of the cells. A new buffer system is developed that facilitates the delivery with greatly reduced cell toxicity. Also provided are methods of using the buffer system to effect the delivery. The polynucleotides may be DNA or RNA, whether single-stranded or double-stranded. The polynucleotides can also be provided in complexes such as RNA-protein complexes.

Accordingly, in one embodiment, the present disclosure provides an aqueous solution, comprising succinate, mannitol, a sugar selected from the group consisting of glucose, sucrose and inositol, glutamine or an analog thereof, and an antioxidant.

In some embodiments, the sugar is D-glucose. In some embodiments, the glutamine or analog thereof is selected from the group consisting of L-alanyl-L-glutamine, L-glutamine, and D-glutamine. In some embodiments, the succinate is sodium succinate, potassium succinate, or magnesium succinate. In some embodiments, the antioxidant is sodium pyruvate or acetylcysteine.

In some embodiments, the solution further comprises Na₂HPO₄ and/or NaH₂PO₄. In some embodiments, the solution further comprises serum.

In some embodiments, the solution includes no or limited NaCl, such as less than 80 mM NaCl, or preferably less than 70 mM, 60 mM, or 50 mM NaCl.

In a particular embodiment, the solution comprises 5 mM to 30 mM sodium succinate, 1 mM to 30 mM mannitol, 3 mM to 30 mM glucose, 50 mg/L to 800 mg/L L-alanyl-L-glutamine, 0.1 mM to 0.6 mM sodium pyruvate, and includes less than 60 mM NaCl.

Another embodiment of the present disclosure provides a method for introducing a polynucleotide into a cell, comprising applying a pulse of electricity to a sample comprising the polynucleotide and the cell in an aqueous solution provided herein.

Another embodiment of the present disclosure provides a method for introducing a polynucleotide into a cell, comprising applying a pulse of electricity to a sample comprising the polynucleotide and the cell in a culture medium that includes less than 80 mM NaCl, or preferably less than 70 mM, 60 mM, or 50 mM NaCl. In some embodiments, the culture medium comprises succinate, mannitol, a sugar, glutamine or an analog thereof, and an antioxidant.

In some embodiments, the polynucleotide is DNA or RNA. In some embodiments, the DNA is single-stranded DNA or double-stranded DNA. In some embodiments, the RNA is siRNA, sgRNA, messenger RNA or double-stranded RNA. In some embodiments, the RNA is provided in an RNA-protein complex.

In some embodiments, the cell is a mammalian cell. In some embodiments, the culture medium further comprises an agent that inhibits the cGAS-STING pathway. In some embodiments, the agent is an siRNA targeting the cGAS or STING protein.

In some embodiments, the method results in entry of the polynucleotide into the nucleus of the cell.

Also provided, in one embodiment, is a method for introducing a biomolecule into a cell, comprising applying a pulse of electricity to a sample comprising the biomolecule and the cell in an aqueous solution having an osmotic pressure greater than 310 mOsmol/kg, wherein the biomolecule is an RNA molecule, a protein, or the combination thereof.

In some embodiments, the aqueous solution has an osmotic pressure greater than 320 mOsmol/kg, 330 mOsmol/kg, 340 mOsmol/kg, or 350 mOsmol/kg. In some embodiments, the osmotic pressure is below 600 osmotic pressure.

In some embodiments, the aqueous solution is as described throughout the present disclosure. In some embodiments, the aqueous solution comprises a non-electrolyte agent for adjusting the osmotic pressure of the aqueous solution. In some embodiments, the non-electrolyte agent is selected from the group consisting of glucose, sucrose, fructose, Mannitol, Sorbitol, lactose, trehalose, Glycerol, PEG300, PEG400, PEG600, Glycine, Proline, Taurine, Betaine, and boric acid. In some embodiments, the non-electrolyte agent is present at concentration of 10 mM to 300 mM, 20 mM to 300 mM, 30 mM to 300 mM, 40 mM to 300 mM, 50 mM to 300 mM, 10 mM to 200 mM, 20 mM to 200 mM, 30 mM to 200 mM, 40 mM to 200 mM, 50 mM to 200 mM, 10 mM to 100 mM, 20 mM to 100 mM, 30 mM to 100 mM, 40 mM to 100 mM, or 50 mM to 100 mM.

In some embodiments, the aqueous solution comprises an electrolyte agent for adjusting the osmotic pressure of the aqueous solution. In some embodiments, the electrolyte agent is selected from the group consisting of NaCl, KCl, MgCl₂, CaCl₂), Ca(NO₃)₂, MgSO₄, and NaHCO₃. In some embodiments, the non-electrolyte agent is present at concentration of 3 mM to 90 mM, 4 mM to 90 mM, 5 mM to 90 mM, 6 mM to 90 mM, 7 mM to 90 mM, 8 mM to 90 mM, 9 mM to 90 mM, 10 mM to 90 mM, 15 mM to 90 mM, 20 mM to 90 mM, 3 mM to 60 mM, 4 mM to 60 mM, 5 mM to 60 mM, 6 mM to 60 mM, 7 mM to 60 mM, 8 mM to 60 mM, 9 mM to 60 mM, 10 mM to 60 mM, 15 mM to 60 mM, 20 mM to 60 mM, 3 mM to 40 mM, 4 mM to 40 mM, 5 mM to 40 mM, 6 mM to 40 mM, 7 mM to 40 mM, 8 mM to 40 mM, 9 mM to 40 mM, 10 mM to 40 mM, 15 mM to 40 mM, 20 mM to 40 mM, 3 mM to 20 mM, 4 mM to 20 mM, 5 mM to 20 mM, 6 mM to 20 mM, 7 mM to 20 mM, 8 mM to 20 mM, 9 mM to 20 mM, 10 mM to 20 mM, or 15 mM to 20 mM.

In some embodiments, the aqueous solution has a pH of 7 to 7.4. In some embodiments, the biomolecule is an siRNA, sgRNA, messenger RNA, double-stranded RNA, antisense RNA, tRNA or RNA aptamer. In some embodiments, the biomolecule is a protein or RNA-protein complex.

Also provided, in one embodiment, is a method for introducing a DNA molecule into a cell, comprising applying a pulse of electricity to a sample comprising the DNA molecule and the cell in an aqueous solution having an osmotic pressure between 270 and 330 mOsmol/kg. In some embodiments, the osmotic pressure inhibits cGAS-STING activation in the cell. In some embodiments, the DNA is double stranded DNA.

### BRIEF DESCRIPTION OF THE DRAWINGS

FIG. 1a-i show that mitigating cGAS-STING activation is essential for non-viral based dsDNA delivery in primary human T cells. a, Fold change of cell number at 72 h post-electroporation over input. 1 μg of plasmid (3.5 kb), double-stranded DNA (1.4 kb), single-stranded DNA (1.4 kb), double-stranded oligonucleotides (99 bp), single-stranded oligonucleotides (99 bp), or MOCK were electroporated into T cells. EP, electroporation. b, Western blot analysis of electroporated T cells showing the activation of STING and its downstream signaling pathway TBK1 and IRF3 in plasmid and dsDNA stimulated cells. cd, RT-qPCR analysis of interferon stimulated genes (c) and inflammation cytokine (d). Cells were collected at 4 h post-electroporation. e, Fold change of cell number at 72 h post-electroporation over input. Stimulated human T cells were first electroporated with Cas9/gRNA as ribonucleoprotein (RNP) targeting AAVS1 locus, STING or cGAS. 72 h post-1^{st} EP, 1 μg of indicated DNA were electroporated into STING KO or cGAS KO or AAVS1-KO cells. dsCAR is a 2.9 kb double-stranded DNA, ssCAR is a 2.9 kb single-stranded DNA. f, Heat-map of GFP efficiency and cell viability. Systematic optimization of electroporation buffer and program using GFP plasmid. Data was collected 24 h after transfection. Recommended commercial electroporation parameter P3-E0115 was boxed black. g, (top) Confocal images of fixed T cells electroporated with indicated buffer and program. Green labels cytoskeleton structure, Blue is nuclei, Red is a cy5-labeled 1.35 kb DNA. (bottom) Quantification of subcellular distribution of DNA. Number of cells quantified is indicated in the bracket. h, FACS analysis of T cells electroporated with 1 μg EF1α-GFP dsDNA (3.2 kb) at indicated EP conditions showed improved transfection efficiency (left), enhanced fluorescent intensity (right) and increased absolute GFP cells (lower) when using BO14-EO138 condition. Data is collected at 72 h post-EP. i, qPCR analysis of T cells electroporated with indicated EP parameters and vehicles. BO14-based electroporation greatly reduced interferon and inflammation regulated genes. Targeted integration of GFP at Rab11a locus (also see FIG. 8a). 1 μg donor dsDNA were used. P value was calculated by student t-test for the entire figure. *p<0.05 **p<0.01 ***p<0.001, n>2 biologically independent samples. Error bars represent mean±s.d.

FIG. 2a-h show that BO14-based nuclear delivery of dsDNA resulted in enhanced knock-in (KI) rate in various applications. a, BO14-based nucleus delivery of 1p g dsDNA templates exhibited enhanced knock-in gene editing efficiency (left) and increased knock-in cell number (right) across a variety of genetic loci. 1p g of dsDNA were used and data were collected at 3 day post-EP for Rab11a, CLTA, and AAVS1 locus, or at 6 days for FBL locus, or at 14 days for TRAC locus due to the episomal expression from donor DNA. Also see FIG. 8. b, Donor modification further improved KI efficiency (left) and knockin cell number (right) in BO14-based delivery system. 0.5 μg dsDNA for Rab11a locus or 1 μg dsDNA for CLTA locus with indicated modification were co-electroporated with respective RNP complex. Data were collected at 3 day post-EP. c, Determine the key components of BO14 transfection system. Five individual components were added back to the basic buffer B both individually or combined as mix. 0.4 ug dsDNA targeting Rab11a locus were chosen and data were measured at 3 day post-EP. d, Non-viral genome targeting a CD19 CAR to the TRAC locus. 3p g RNP complex and a 2.9 kb CD19CAR dsDNA template with indicated dose were co-electroporated into primary human T cell. FACS analysis of CAR expression and cell count was performed at 5 days post-EP. Also see FIG. 9. e, Cell killing analysis of CD19-CAR T cells incubated with CD19+Nalm6 tumor cells or CD19^{-/-}Nalm6 cells at indicated ratio. f, Antigen-specific cytokine production in BO14-generated CAR-T cells and in AAV1-generated CAR-T cells. Negative control represents CAR-T cells incubated with CD19^{-/-}Nalm6 cell line. See also FIG. 9c. g, NCG mice were inoculated with 2.5×10⁵ CD19+Nalm6 cells and followed by 5×10⁵ CAR-T cells. Tumor burden measured as bioluminescent signal were quantified (n=2 per group). h, Kaplan-Meier analysis of survival of mice treated with different CAR-T cells or PBS control (Ctrl). (n=7 per group). P value was calculated by t-test for the entire figure. *p<0.05 **p<0.01 ***p<0.001, n>3 biologically independent samples. Error bars represent mean±s.d.

FIG. 3a-b show that cGAS/STING pathway is critical for DNA sensing in primary T cells. a, Validation of effective knockdown of cGAS and STING by two sgRNAs in primary human T cells. b, qPCR analysis of cGAS/STING downstream pathway. Cells were collected 4 h post electroporation.

FIG. 4a-d show that STING inhibitor showed little effect on DNA induced immune response in T cells. a-b, Human primary T cells were pretreated with 1 μM H151, a STING inhibitor, for 16 h prior to electroporation. Cell number were counted at 72 h post-EP. c, qPCR analysis of ISG56 and IFNB1 showed persist gene expression upon STING inhibitor treatment. DMSO, dimethyl sulfoxide, is the vehicle control. d, Human monocyte cell line THP-1 were pretreated with H151 at indicated dose prior to HSV-1 stimulation. qPCR analysis of ISG56 and IFN-β showed dose response towards H151, the STING inhibitor.

FIG. 5a-c show that double electroporation is not viable for T cell engineering. a, Western blot analysis showed effective knockdown of STING by siRNA. b, Viability analysis of cells treated with siSTING prior to electroporation with plasmid DNA. c, Cell count were performed 3 days post-EP and fold change is calculated by dividing with input cell. Primary human T cell is tolerant with single electroporation as evidenced by 5-fold cell growth over three days whereas double electroporation showed no cell growth, indicating double electroporation is not viable for T cell engineering. n=3 biologically independent samples.

FIG. 6a-b show validation of screened EP conditions. a, Based on the initial screen, two buffers BO14 and AO11 were validated with multiple T cell donors. 0.4 μg pMAX plasmid were electroporated with indicated buffer and EP program. GFP positive cells and median fluorescence intensity were counted and measured 1 day post-EP. Four different donor T cells were tested. b, Time course analysis of transfection efficiency. 0.4 μg pMAX plasmid were electroporated with indicated buffer and EP program. Transfection efficiency was measured 1, 3 and 5 day post-EP. Four different donor T cells were tested.

FIG. 7a-b show that BO14-mediated nuclear delivery help mitigate DNA induced immune response in primary human T cells. a, qPCR analysis of T cells electroporated with indicated EP conditions and vehicles. BO14 buffer mediated electroporation greatly reduced interferon and inflammation responsive genes. b, Western blot analysis of STING activation. Cells were collected 4 h post-EP for analysis.

FIG. 8a-c show that BO14-mediated nuclear delivery enhanced large fragment targeted insertion across multiple locus. a, GFP sequence were inserted into gene exon to make a fusion protein. b, EF1α promoter driven GFP is inserted into the TRAC locus. c, GFP sequence was inserted into the intron of AAVS1 locus. Representative FACS plot of target insertion are listed below. 1 μg of dsDNA were used and data were collected at 3 days post-EP for Rab11a, CTLA, and AAVS1 locus, or at 6 days for FBL locus or at 14 days for TRAC locus.

FIG. 9a-c show that BO14-mediated nuclear delivery enhanced CAR-T cell generation. a, CD19BBz CAR knock-in strategy at TRAC locus. a 15 bp truncated Cas9 target sequence (tCTS) was added at the 5′ ends of the HDR template. dsDNA were 5′ modified with 5′ phosphorothioate (PS) and C6-polyethylene glycol 10 (PEG). b, FACS blot showing effective CAR expression (˜32%) and up to 97% TCRα knockout. c, Gating strategy and raw FACS blots for FIG. 2f.

FIG. 10A-E shows that the optimized EP protocol exhibited enhanced plasmid delivery in 293T Cell line. A, Systematic optimization of electroporation process using GFP plasmids in 293T cell line. Analysis of GFP expression and cell viability 24 h after transfection by flow cytometry. Data are from one experiment. B, Validation of plasmid delivery in 293T cells. Cell viability and transfection efficiency (GFP expression and MFI) were evaluated 24 h after transfection by flow cytometry. TSF-CM130 represents the commercial recommended (Lonza) SF buffer and EP program CM130. SF-EW113 represents commercial SF buffer paired with EW113 EP program. Inhouse EP buffer was coted BO11. Data are shown as mean±SD (n=3) from three independent experiments. C, (top) Inhouse EP buffer showed enhanced gene knockout efficiency using CRISPR plasmid pX330 targeting EGFP locus. (bottom) Example of KO efficiency of px330 plasmid (1.0 μg dose) compared with commercial Kit 7 days after transfection. Knockout efficiency was measured by EGFP-negative cells 7 days post electroporation. Data are presented as mean±SD (n=5) and representative of five independent experiments. D, (top) Comparable RNP delivery to commercial kit. Delivery of CAs9/gRNA as RNP targeting EGFP by inhouse EP protocols were compared with commercial Kit in 293T cell line. (bottom) Example of KO efficiency of RNP (5:1 RNA/Cas9 ratio and 1.5 μg Cas9) compared with commercial Kit 7 days after transfection. Knockout efficiency was measured by EGFP-negative cells 7 days post electroporation. Data are presented as mean±SD (n=5) and representative of five independent experiments. E, Comparison of KO efficiency and cell viability 72 h after RNP transfection targeting VEGFA using optimized inhouse EP protocols were compared with commercial Kit in 293T cell line. Data are presented as mean±SD (n=3) and representative of three independent experiments. *, P<0.05; **, P<0.01; *** P<0.001; by one-way ANOVA.

FIG. 11A-C show that the optimized electroporation protocols and efficient delivery of CRISPR in cell lines. (A) Cell viability and transfection efficiency obtained by optimized inhouse EP protocols were compared with commercial Kit in U-2 OS, jurkat, K562 and Raw264.7 cell lines. Cell viability and transfection efficiency (GFP expression and MFI) were evaluated 24 h after transfection by flow cytometry. Data are shown as mean±SD from three or more independent experiments. (B and C) KO efficiency, indel rate and cell viability 2-4 days after nucleofection of px330 plasmid (B) and RNP (C) targeting TRAC by optimized inhouse EP protocols were compared with commercial Kit in jurkat cell line. Data are presented as mean SD and representative of three or more independent experiments.

FIG. 12A-F show that the optimize electroporation and test for RNP delivery in mouse primary cells. (A and B) Enhanced Plasmid delivery (A) and RNP delivery (B) via BO based buffer system in primary mouse T cell. P3 represents lonza commercial buffer and DN100 is the recommended electroporation program. GFP plasmid was used in A and Cas9/gRNA targeting CD90 was used in B. Cell viability, transfection efficiency (GFP expression and MFI) were evaluated 24 h after transfection by flow cytometry. KO efficiency as measured by CD90-negative T cells and cell viability 72 h after nucleofection of RNPs targeting CD90 by flow cytometry in mouse primary T cells. Data are shown as mean±SD from three independent experiments. (C) Enhanced Plasmid delivery in primary mouse macrophage. P2-DO100 represents Lonza recommended buffer and EP program. Data are shown as mean±SD from four independent experiments. (D-F) Enhanced mRNA delivery (D, F) and RNP delivery (E) via BO based buffer system in primary human T cell. 0.5 ug GFP mRNA was used in D and data was collected 24 h post EP. KO efficiency as measured by TRAC-negative T cells and cell viability 72 h (96 h for Cas9 mRNA/sgRNA) after RNP (E) and Cas9 mRNA/sgRNA (F) transfection.

FIG. 13A-B show the effects of the enhanced HDR in buffer B based delivery system. (A-B) Cas9 mediated HDR target RAB11a locus in Jurkat cell line (A) and 293T (B). 1 ug dsDNA donor and 3 ug RNP targeting Rab11a locus were electroporated into cells and data were collected by FACS 72 h post EP.

FIG. 14a-d shows optimization and testing of EP of mRNA into human hematopoietic stem and progenitor cell (HSPCs). a and b, systematic optimization of electroporation (EP) process using GFP mRNA in human CD34+ HSPCs. Analysis of median fluorescence intensity (MFI) and number of GFP positive cells 12 h post-EP by flow cytometry. Data are from the average of two or three repeated experiment. Black box indicates commercial standard from Lonza. BO14, BO11, or A041 represents different optimized electroporation buffer. c and d, New electroporation system of buffer BO11 with DG135 electroporation program showed three-fold increase in fluorescence intensity (c) and comparable transfected cells (d) in human HSPCs.

FIG. 15 presents schematic illustration of commonly used delivery methods in CAR-T generation. Conventional electroporation triggered severe STING activation and led to strong cell toxicity. cGAS or STING knockout could help prevent STING activation and promote cell survival. The new identified electroporation condition functions to enhance nuclear delivery, thereby attenuating the activation of cGAS-STING and thus promoting cell survival and targeted insertion.

FIG. 16A-D show that isotonic osmotic pressure is optimal for dsDNA delivery.

FIG. 17A-B show that higher osmotic pressures improved mRNA delivery in Jurkat cells.

FIG. 18A-C show that higher osmotic pressures improved mRNA delivery in human primary cells.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Electroporation Buffers

Generally, introduction of foreign polynucleotides into a target cell, e.g., through electroporation, has low efficiency. When the target cell is a mammalian cell, another major challenge is cellular toxicity induced by the foreign polynucleotides. The mechanism of such cellular toxicity is not well-understood. It is contemplated that the presence of cytosolic double-stranded DNA per se triggers immune response. Accordingly, to reduce or avoid cellular toxicity, it is desirable to deliver the foreign polynucleotides directly into the cell nuclei. Compared to delivery to the cytoplasm, however, direct delivery to the nucleus has even lower efficiency.

The instant inventors explored the mechanism of cellular toxicity induced by DNA electroporation. When plasmid or double stranded DNA (dsDNA) was transfected into T cells, strong activation was observed for phosphorylated stimulator of interferon response cGAMP interactor 1 protein (p-STING), and its downstream signaling molecules, phosp-TBK1 (p-TBK1) and phosp-IRF3 (p-IRF3). Also observed was production of inflammatory cytokines and type I interferon (IFN) gene expression, downstream of cyclic GMP-AMP synthases (cGAS)-STING signaling.

To further confirm that the cellular toxicity is mediated by the cGAS-STING signaling pathway, the inventors knocked out cGAS and STING expression with the Cas-CRISPR technology. The results show that cGAS or STING knockout cells exhibited significantly increased cell viability, as compared to control cells, upon dsDNA electroporation. These results, therefore, demonstrate that cGAS-STING mediated immune activation is the major cause of electroporation-induced cell death, and inhibition of this signaling pathway can reduce the toxicity, thereby improving electroporation efficiency.

cGAS is a cytosolic DNA sensor and does not react with nucleus DNA. Accordingly, the instant inventor designed another strategy to reduce cellular toxicity, by promoting nuclear delivery of polynucleotides. Nuclear delivery would bypass the surveillance of cytosolic cGAS. To this end, the inventors performed high throughput screening of electroporation buffers, and identified buffer B. When combined with the commercial cell culture medium, Opti-MEM™ medium at different ratios, buffer B achieved the highest nuclear delivery of DNA in various types of mammalian cells. Notably, buffer B included succinate and mannitol, which were determined to be responsible for its exceptional ability to promote nuclear uptake of the foreign polynucleotides. The inventors further determined that, within Opti-MEM, the sugar (e.g., glucose) and glutamine analog (L-alanyl-L-glutamine) are responsible for the success. Also discovered, unexpectedly, is that high salt concentrations (e.g., 90 mM NaCl of buffer A) can be detrimental to the nuclear uptake of certain forms of polynucleotides.

Opti-MEM™ medium is a reduced serum medium commercially available from Fisher Scientific. It is an improved Minimal Essential Medium (MEM) that allows for a reduction of Fetal Bovine Serum supplementation by at least 50% with no change in growth rate or morphology. The main ingredients relevant to the present technology are provided in Table 1. In particular, the Opti-MEM™ medium includes i-Inositol, Ca(NO₃)₂·4H₂O, sodium pyruvate, D-glucose and GlutaMAX (L-alanyl-L-glutamine).

In accordance with one embodiment of the present disclosure, provided is an aqueous solution that includes succinate, mannitol, a sugar, glutamine or an analog thereof, and/or an antioxidant. In some embodiments, the sugar is selected from glucose, sucrose and inositol.

In accordance with another embodiment of the present disclosure, provided is an aqueous solution that includes less than 80 mM NaCl, or preferably less than 70 mM, 60 mM, 55 mM, 50 mM, 45 mM, 40 mM, 35 mM, 30 mM, 25 mM or 20 mM NaCl, which is useful for electroporation. In some embodiments, the aqueous solution includes a sugar, glutamine or an analog thereof, and an antioxidant. In some embodiments, the aqueous solution further includes succinate and/or mannitol. In some embodiments, the sugar is selected from glucose, sucrose and inositol.

Succinate, such as sodium succinate, potassium succinate, and magnesium succinate, is contemplated to be able to increase cell viability. In some embodiments, the aqueous solution includes at least 5 mM, 10 mM, 15 mM, 20 mM, or 25 mM succinate (e.g., sodium succinate). In some embodiments, the aqueous solution includes no more than 50 mM, 45 mM, 40 mM, 35 mM, 30 mM, 25 mM, 20 mM, 15 mM or 10 mM succinate (e.g., sodium succinate).

Mannitol is contemplated to promote homology directed DNA repair. The instant results show that mannitol improves transfer of foreign polynucleotides through the cytoplasm into the nuclei. In some embodiments, the aqueous solution includes at least 5 mM, 10 mM, 15 mM, 20 mM, or 25 mM mannitol. In some embodiments, the aqueous solution includes no more than 50 mM, 45 mM, 40 mM, 35 mM, 30 mM, 25 mM, 20 mM, 15 mM or 10 mM mannitol.

The sugar included in the aqueous solution is contemplated to optimize the osmolality of the solution, for optimized electroporation and nuclear uptake. In some embodiments, the sugar is selected from glucose, sucrose and inositol. In some embodiments, the aqueous solution includes at least 5 mM, 10 mM, 15 mM, 20 mM, or 25 mM sugar. In some embodiments, the aqueous solution includes no more than 50 mM, 45 mM, 40 mM, 35 mM, 30 mM, 25 mM, 20 mM, 15 mM, 10 mM, or 5 mM sugar. In some embodiments, the aqueous solution includes at least 5 mM, 10 mM, 15 mM, 20 mM, or 25 mM D-glucose. In some embodiments, the aqueous solution includes no more than 50 mM, 45 mM, 40 mM, 35 mM, 30 mM, 25 mM, 20 mM, 15 mM, 10 mM, or 5 mM D-glucose. In some embodiments, the aqueous solution includes at least 5 mM, 10 mM, 15 mM, 20 mM, or 25 mM sucrose. In some embodiments, the aqueous solution includes no more than 50 mM, 45 mM, 40 mM, 35 mM, 30 mM, 25 mM, 20 mM, 15 mM, 10 mM, or 5 mM sucrose. In some embodiments, the aqueous solution includes at least 5 mM, 10 mM, 15 mM, 20 mM, or 25 mM inositol. In some embodiments, the aqueous solution includes no more than 50 mM, 45 mM, 40 mM, 35 mM, 30 mM, 25 mM, 20 mM, 15 mM, 10 mM, or 5 mM inositol.

Glutamine, or analogs, is contemplated to promote cell health and vitality. In some embodiments, the glutamine or analog thereof is selected from the group consisting of L-alanyl-L-glutamine, L-glutamine, and D-glutamine. In some embodiments, the aqueous solution includes at least 50 mg/L, 100 mg/L, 110 mg/L, 120 mg/L, 130 mg/L, 140 mg/L, 150 mg/L, 160 mg/L, 170 mg/L, 180 mg/L, 190 mg/L, 200 mg/L, 210 mg/L, 220 mg/L, 230 mg/L, 240 mg/L, 250 mg/L, 260 mg/L, 270 mg/L, 280 mg/L, 290 mg/L, or 300 mg/L glutamine or an analog thereof. In some embodiments, the aqueous solution includes no more than 1000 mg/L, 900 mg/L, 850 mg/L, 800 mg/L, 750 mg/L, 700 mg/L, 650 mg/L, 600 mg/mL, 650 mg/L, 600 mg/L, 550 mg/L, 500 mg/L, 480 mg/L, 460 mg/L, 440 mg/L, 420 mg/L, 400 mg/L, 380 mg/L, 360 mg/L, 340 mg/L, 320 mg/L, 300 mg/L, 280 mg/L, 260 mg/L, 240 mg/L, 220 mg/L, 200 mg/L, 180 mg/L, 160 mg/L, 140 mg/L, 120 mg/L, or 100 mg/L glutamine or an analog thereof. In some embodiments, the aqueous solution includes at least 50 mg/L, 100 mg/L, 150 mg/L, 200 mg/L, 250 mg/L, 300 mg/L, 350 mg/L, 400 mg/L, 450 mg/L, 500 mg/L, 550 mg/L, 600 mg/L, 650 mg/L, 700 mg/L, 750 mg/L, or 800 mg/L L-alanyl-L-glutamine. In some embodiments, the aqueous solution includes no more than 1000 mg/L, 900 mg/L, 850 mg/L, 800 mg/L, 750 mg/L, 700 mg/L, 650 mg/L, 600 mg/mL, 550 mg/L, 500 mg/L, 480 mg/L, 460 mg/L, 440 mg/L, 420 mg/L, 400 mg/L, 380 mg/L, 360 mg/L, 340 mg/L, 320 mg/L, 300 mg/L, 280 mg/L, 260 mg/L, 240 mg/L, 220 mg/L, 200, or 200 mg/L L-alanyl-L-glutamine.

An “antioxidant” refers to a molecule capable of slowing or preventing the oxidation of other molecules. Oxidation is a chemical reaction that transfers electrons from a substance to an oxidizing agent. Oxidation reactions can produce free radicals, which start chain reactions that destabilize the protein therapeutics and ultimately affect the product activity. Antioxidants terminate these chain reactions by removing free radical intermediates, and inhibit other oxidation reactions by being oxidized themselves. As a result, antioxidants are often reducing agents, chelating agent and oxygen scavengers such as pyruvate, acetylcysteine, citrate, EDTA, DPTA, thiols, ascorbic acid or polyphenols. Non-limiting examples of antioxidants include ascorbic acid (AA, E300), thiosulfate, methionine, tocopherols (E306), propyl gallate (PG, E310), tertiary butylhydroquinone (TBHQ), butylated hydroxyanisole (BHA, E320) and butylated hydroxytoluene (BHT, E321).

In some embodiments, the antioxidant is pyruvate (e.g., sodium pyruvate). In some embodiments, the antioxidant is acetylcysteine. In some embodiments, the aqueous solution includes at least 0.1 mM, 0.15 mM, 0.2 mM, 0.25 mM, 0.3 mM, 0.4 mM or 0.5 mM sodium pyruvate. In some embodiments, the aqueous solution includes no more than 0.6 mM, 0.5 mM, 0.4 mM, 0.35 mM, 0.3 mM, 0.25 mM, 0.2 mM, or 0.15 mM sodium pyruvate. In some embodiments, the aqueous solution includes at least 0.1 mM, 0.15 mM, 0.2 mM, 0.25 mM, 0.3 mM, 0.4 mM or 0.5 mM acetylcysteine. In some embodiments, the aqueous solution includes no more than 0.6 mM, 0.5 mM, 0.4 mM, 0.35 mM, 0.3 mM, 0.25 mM, 0.2 mM, or 0.15 mM acetylcysteine.

In some embodiments, the aqueous solution further includes Na₂HPO₄ and/or NaH₂PO₄. Suitable concentrations for the Na₂HPO₄ and/or NaH₂PO₄ in the solution may be 20 mM to 200 mM, 30 mM to 180 mM, 40 mM to 160 mM, 50 mM to 150 mM, 60 mM to 130 mM, 70 mM to 110 mM, 80 mM to 95 mM, without limitation.

In some embodiments, the aqueous solution further includes KCl, MgCl₂ or MgSO₄, Ca(NO₃)₂, amino acids, phenol red, and/or HEPES (4-(2-hydroxyethyl)-1-piperazineethanesulfonic acid). In some embodiments, the aqueous solution has a pH of 6.8 to 7.6, 6.9 to 7.5, 7 to 7.4, 7.1 to 7.3, 7.15 to 7.25, or about 7.3, without limitation. In some embodiments, the aqueous solution further includes serum.

In some embodiments, the aqueous solution includes no or limited NaCl, such as less than 80 mM NaCl, or less than 70 mM, 60 mM, 55 mM, 50 mM, 45 mM, 40 mM, 35 mM, 30 mM or 25 mM NaCl.

In some embodiments, the aqueous solution includes 5 mM to 30 mM sodium succinate, 1 mM to 30 mM mannitol, 3 mM to 30 mM glucose, 50 mg/L to 900 mg/L L-Glutamine or L-alanyl-L-glutamine, 0.1 mM to 0.6 mM sodium pyruvate, and includes less than 60 mM NaCl.

In some embodiments, the aqueous solution includes 8 mM to 25 mM sodium succinate, 3 mM to 25 mM mannitol, 5 mM to 25 mM glucose, 100 mg/L to 700 mg/L L-Glutamine or L-alanyl-L-glutamine, 0.2 mM to 0.5 mM sodium pyruvate, and includes less than 50 mM NaCl.

In some embodiments, the aqueous solution includes 10 mM to 20 mM sodium succinate, 5 mM to 20 mM mannitol, 8 mM to 20 mM glucose, 200 mg/L to 500 mg/L L-Glutamine or L-alanyl-L-glutamine, 0.3 mM to 0.4 mM sodium pyruvate, and includes less than 40 mM NaCl.

### Osmotic Pressures of Electroporation Buffers for RNA and Proteins

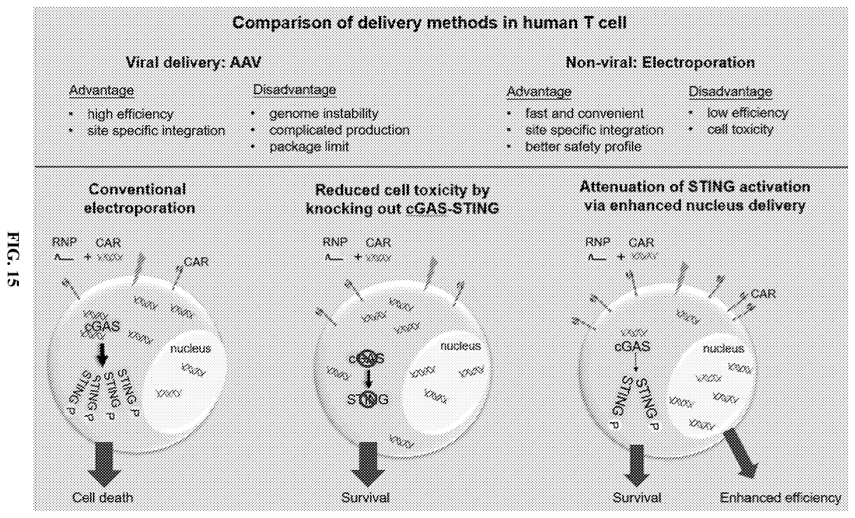

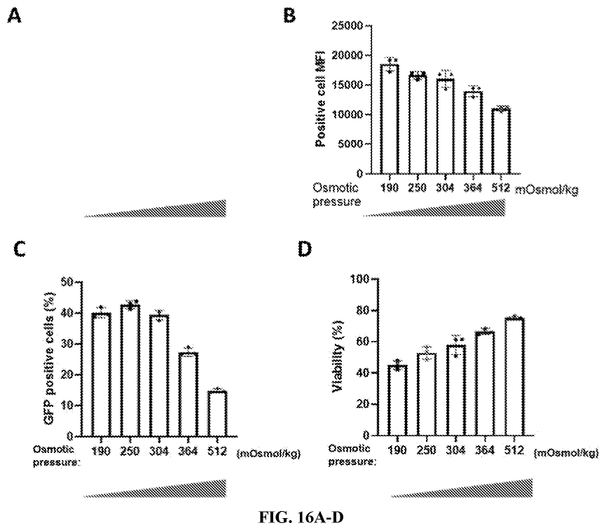

In another surprising discovery, the instant inventors demonstrated that the osmotic pressure of the electroporation buffer has significant impact on the delivery of large molecules. For double stranded DNA (dsDNA), for instance, higher osmotic pressure tends to reduce the transfection efficiency (FIG. 16B-C). Lower osmotic pressure, however, is more detrimental to target cell viability (FIG. 16D). An optimal osmotic pressure, therefore, is at about the isotonic point (around 300 mOsmol/kg), see, e.g., FIG. 16A.

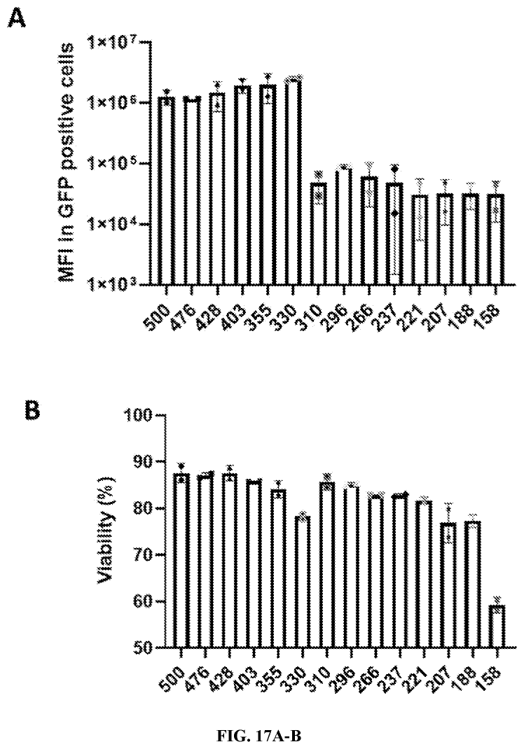

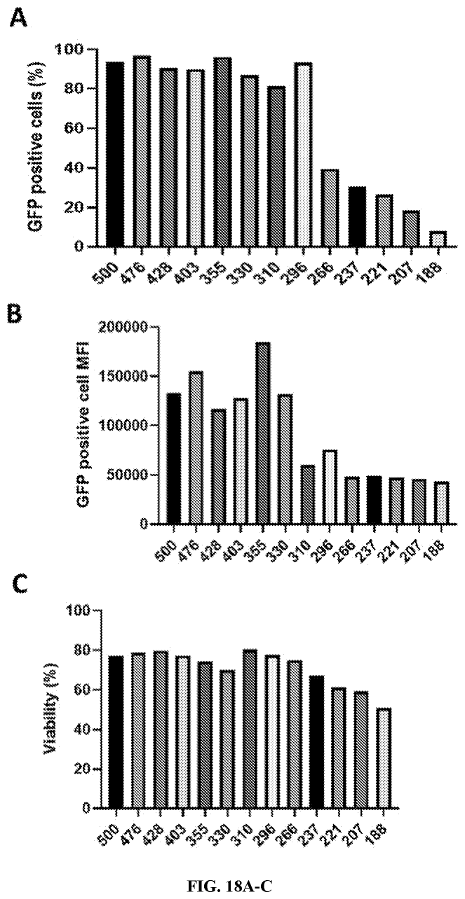

Such an observation to dsDNA, however, did not hold for RNA. For both cultured Jurkat cells and primary human T cells, higher osmolality of the electroporation buffers (e.g., above 330 mOsmol/kg) was beneficial to both transfection efficiency and target cell viability (FIG. 17-18). It is contemplated that such a correlation is also applicable to delivery of proteins to cells through electroporation.

In accordance with one embodiment of the present disclosure, therefore, provided is a method for introducing an RNA or protein into a cell, comprising applying a pulse of electricity to a sample that includes the RNA or protein and a target cell, in an aqueous solution having an osmotic pressure that is higher than 300 mOsmol/kg, or higher than 310 mOsmol/kg, 320 mOsmol/kg, 330 mOsmol/kg, 340 mOsmol/kg, or 350 mOsmol/kg.

In some embodiments, the solution has an osmotic pressure that is from 310 mOsmol/kg to 600 mOsmol/kg. In some embodiments, the solution has an osmotic pressure that is from 310 mOsmol/kg to 600 mOsmol/kg, 550 mOsmol/kg, 500 mOsmol/kg, 450 mOsmol/kg, 400 mOsmol/kg, or 350 mOsmol/kg. In some embodiments, the solution has an osmotic pressure that is from 320 mOsmol/kg to 600 mOsmol/kg, 550 mOsmol/kg, 500 mOsmol/kg, 450 mOsmol/kg, 400 mOsmol/kg, or 350 mOsmol/kg. In some embodiments, the solution has an osmotic pressure that is from 330 mOsmol/kg to 600 mOsmol/kg, 550 mOsmol/kg, 500 mOsmol/kg, 450 mOsmol/kg, 400 mOsmol/kg, or 350 mOsmol/kg. In some embodiments, the solution has an osmotic pressure that is from 340 mOsmol/kg to 600 mOsmol/kg, 550 mOsmol/kg, 500 mOsmol/kg, 450 mOsmol/kg, 400 mOsmol/kg, or 350 mOsmol/kg.

In some embodiments, the solution has an osmotic pressure that is from 350 mOsmol/kg to 600 mOsmol/kg, 550 mOsmol/kg, 500 mOsmol/kg, 450 mOsmol/kg, or 400 mOsmol/kg. In some embodiments, the solution has an osmotic pressure that is from 360 mOsmol/kg to 600 mOsmol/kg, 550 mOsmol/kg, 500 mOsmol/kg, 450 mOsmol/kg, or 400 mOsmol/kg. In some embodiments, the solution has an osmotic pressure that is from 370 mOsmol/kg to 600 mOsmol/kg, 550 mOsmol/kg, 500 mOsmol/kg, 450 mOsmol/kg, or 400 mOsmol/kg. In some embodiments, the solution has an osmotic pressure that is from 380 mOsmol/kg to 600 mOsmol/kg, 550 mOsmol/kg, 500 mOsmol/kg, 450 mOsmol/kg, or 400 mOsmol/kg. In some embodiments, the solution has an osmotic pressure that is from 390 mOsmol/kg to 600 mOsmol/kg, 550 mOsmol/kg, 500 mOsmol/kg, 450 mOsmol/kg, or 400 mOsmol/kg. In some embodiments, the solution has an osmotic pressure that is from 400 mOsmol/kg to 600 mOsmol/kg, 550 mOsmol/kg, 500 mOsmol/kg, or 450 mOsmol/kg.

A suitable solution can be prepared by admixing a commonly used or presently disclosure buffer (e.g., BO14, BO11, B-ICSGG, and B-2xICSGG and their derivatives) with an electrolyte or a non-electrolyte, such as an organic molecule (e.g., sugar, amino acid, polymer).

An example buffer suitable for preparing an aqueous solution with the desired osmotic pressure includes succinate, mannitol, a sugar selected from the group consisting of glucose, sucrose and inositol, glutamine or an analog thereof, and an antioxidant.

In some embodiments, the sugar is D-glucose. In some embodiment, the glutamine or analog thereof is selected from the group consisting of L-alanyl-L-glutamine, L-glutamine, and D-glutamine. In some embodiments, the succinate is sodium succinate, potassium succinate, or magnesium succinate. In some embodiments, the antioxidant is sodium pyruvate or acetylcysteine. In some embodiments, the buffer further includes Na₂HPO₄ and NaH₂PO₄. In some embodiments, the buffer further includes serum. In some embodiments, the buffer includes less than 80 mM NaCl, or preferably less than 70 mM, 60 mM, or 50 mM NaCl.

Various additional examples of the buffer are described in the section above, under heading “Electroporation Buffers.” In a particular example, the buffer includes 5 mM to 30 mM sodium succinate, 1 mM to 30 mM mannitol, 3 mM to 30 mM glucose, 50 mg/L to 900 mg/L L-Glutamine or L-alanyl-L-glutamine, 0.1 mM to 0.6 mM sodium pyruvate, and includes less than 60 mM NaCl.

In some embodiments, the osmotic pressure of the buffer can be adjusted with a non-electrolyte agent, such as those listed in Table A below.

TABLE A
Example Non-Electrolyte Agent for Adjusting Osmotic Pressure

| Agent (non-electrolyte) | Example concentrations |
|---|---|
| glucose | 10 mM-300 mM |
| sucrose | 10 mM-300 mM |
| fructose | 10 mM-300 mM |
| Mannitol | 10 mM-200 mM |
| Sorbitol | 10 mM-300 mM |
| lactose | 10 mM-200 mM |
| trehalose | 10 mM-300 mM |
| Glycerol | 10 mM-300 mM |
| PEG300 | 10 mM-300 mM |
| PEG400 | 10 mM-300 mM |
| PEG600 | 10 mM-200 mM |
| Glycine | 10 mM-300 mM |
| Proline | 10 mM-300 mM |
| Taurine | 10 mM-200 mM |
| Betaine | 10 mM-300 mM |
| boric acid | 10 mM-300 mM |

Example concentrations of these agents are provided in Table A. Generally, at such concentrations, the osmotic pressure of the buffer can be adjusted within the range of 300-600 mOsmol/kg. The actual concentration used can be determined based on the desired osmotic pressure, which can be measured experimentally.

In some embodiments, the concentration of the non-electrolyte agent is 10 mM to 200 mM or 300 mM. In some embodiments, the concentration of non-electrolyte agent is 20 mM to 200 mM or 300 mM, 30 mM to 200 mM or 300 mM, 40 mM to 200 mM or 300 mM, 50 mM to 200 mM or 300 mM, 60 mM to 200 mM or 300 mM, 70 mM to 200 mM or 300 mM, 80 mM to 200 mM or 300 mM, 90 mM to 200 mM or 300 mM, or 100 mM to 200 mM or 300 mM.

In some embodiments, the concentration of the non-electrolyte agent is 10 mM to 100 mM. In some embodiments, the concentration of non-electrolyte agent is 20 mM to 100 mM, 30 mM to 100 mM, 40 mM to 100 mM, 50 mM to 100 mM, 60 mM to 100 mM, 70 mM to 100 mM, 80 mM to 100 mM, or 90 mM to 100 mM.

In some embodiments, the osmotic pressure of the buffer can be adjusted with a non-electrolyte agent, such as those listed in Table B below.

TABLE B
Example Electrolyte Agent for Adjusting Osmotic Pressure

| Agent (electrolyte) | Example concentrations |
|---|---|
| NaCl | 5 mM-90 mM |
| KCl | 5 mM-90 mM |
| MgCl₂ | 3 mM-40 mM |
| CaCl₂ | 3 mM-40 mM |
| Ca(NO3)₂ | 3 mM-40 mM |
| MgSO₄ | 5 mM-40 mM |
| NaHCO₃ | 5 mM-90 mM |

Example concentrations of these agents are provided in Table B. Generally, at such concentrations, the osmotic pressure of the buffer can be adjusted within the range of 300-480 mOsmol/kg. The actual concentration used can be determined based on the desired osmotic pressure, which can be measured experimentally.

In some embodiments, the concentration of the electrolyte agent is 3 mM to 40 mM or 90 mM. In some embodiments, the concentration of the electrolyte agent is 4 mM to 40 mM or 90 mM, 5 mM to 40 mM or 90 mM, 6 mM to 40 mM or 90 mM, 7 mM to 40 mM or 90 mM, 8 mM to 40 mM or 90 mM, 9 mM to 40 mM or 90 mM, 10 mM to 40 mM or 90 mM, 12 mM to 40 mM or 90 mM, 14 mM to 40 mM or 90 mM, 15 mM to 40 mM or 90 mM, 17 mM to 40 mM or 90 mM, 19 mM to 40 mM or 90 mM, or 20 mM to 40 mM or 90 mM.

In some embodiments, the concentration of the electrolyte agent is 3 mM to 30 mM. In some embodiments, the concentration of the electrolyte agent is 4 mM to 30 mM, 5 mM to 30 mM, 6 mM to 30 mM, 7 mM to 30 mM, 8 mM to 30 mM, 9 mM to 30 mM, 10 mM to 30 mM, 12 mM to 30 mM, 14 mM to 30 mM, 15 mM to 30 mM, 17 mM to 30 mM, 19 mM to 30 mM, or 20 mM to 30 mM.

In some embodiments, the solution has a pH of 6 to 8, 6.1 to 8, 6.2 to 8, 6.3 to 8, 6.4 to 7.9, 6.5 to 7.8, 6.6 to 7.7, 6.8 to 7.6, 6.9 to 7.5, 7 to 7.4, 7.1 to 7.3, 7.15 to 7.25, or about 7.3, without limitation.

In some embodiment, the method is used to deliver an RNA molecule to a target cell, including prokaryotic cells and eukaryotic cells, which may be mammalian cells such as an animal cell including a human cell. In some embodiments, the target cell is an immune cell such as a T cell or an NK cell. In some embodiments, the RNA is an mRNA molecule, or an siRNA, shRNA, rRNA or tRNA. In some embodiments, the method is used to deliver a protein to a target cell. In some embodiments, the method is used to deliver a RNA-protein complex, without limitation.

Likewise, methods for introducing a DNA molecule to a target cell are also provided, wherein the osmotic pressure is provided as close to isotonic (e.g., between 270 and 330 mOsmol/kg, or between 280 and 320 mOsmol/kg, or between 290 and 310 mOsmol/kg, or between 295 and 305 mOsmol/kg). The isotonic osmotic pressure may help mitigate cGAS-STING signaling/activation in the cell that is activated by the DNA molecule. In some embodiments, the DNA is dsDNA. Compositions and methods useful for providing the desired osmotic pressure are described above.

### Electroporation Methods

Methods for introducing polynucleotides into target cells, e.g., through electroporation, are also provided. In one embodiment, a method is provided for introducing a polynucleotide into a cell. The method, in some embodiments, entails applying a pulse of electricity to a sample that includes the polynucleotide and the cell in an aqueous solution provided herein.

As demonstrated in the experimental examples, various types of polynucleotides can be effectively delivered to the nuclei of cells with the present technology. In some embodiments, the polynucleotide is DNA or RNA. In some embodiments, the DNA is single-stranded DNA or double-stranded DNA. In some embodiments, the RNA is siRNA, sgRNA, or single- or double-stranded RNA. In some embodiments, the RNA is provided in an RNA-protein complex.

In some embodiments, the cell is a mammalian cell. In some embodiments, the cell is an immune cell, such as a natural killer (NK) cell, a T cell, a macrophage, or a monocyte, without limitation.

In some embodiments, the method entails applying a pulse of electricity to a sample that includes the polynucleotide and the cell which has been treated with an agent, or in the presence of an agent that inhibits the cGAS-STING pathway. In some embodiments, the method is carried out in an aqueous solution of the present disclosure.

In some embodiments, the agent is an siRNA or antibody targeting the cGAS or STING protein. Methods are well known for preparing inhibitory RNA and antibodies that can be effectively delivered to a cell to effect their inhibitory functions. In some embodiments, the agent is removed at some time following the electroporation, such as after 1 hour, 2 hours, 6 hours, 12 hours, a day, 2 days, 3 days, 7 days, 14 days, a month, or two months, without limitation.

Pulses of electricity can be generated with devices and systems that are commercially available, including those tested herein, such as 4D-nucleofector (Lonza). Each device is equipped with suitable electroporation programs, which can be further tuned for specific applications. The use and tuning are within the capability of skilled artisans.

In some embodiments, the method results in entry of the polynucleotide into the nucleus of the cell.

### EXAMPLES

The following examples are included to demonstrate specific embodiments of the disclosure. It should be appreciated by those of skills in the art that the techniques disclosed in the examples which follow represent techniques to function well in the practice of the disclosure, and thus can be considered to constitute specific modes for its practice. However, those of skills in the art should, in light of the present disclosure, appreciate that many changes can be made in the specific embodiments which are disclosed and still obtain a like or similar result without departing from the spirit and scope of the disclosure.

### Example 1: Enhanced Genome Editing Efficiency by Mitigating cGAS-STING Activation in Non-Viral Nucleus-Targeted DNA Delivery

This example demonstrates that DNA delivery-induced toxicity in primary human T cell is mediated by cGAS-STING. To reduce DNA delivery-induced toxicity in T cells, this example systematically investigated electroporation conditions and identified a buffer composition which could significantly bypass the cytosolic cGAS-STING surveillance via enhanced DNA nucleus delivery. The identified BO14-buffer led to a five-fold increase of reprogrammed CAR-T cells compared with commercial standard after electroporation of DNA template. In addition, BO14-buffer generated CAR-T showed more potent tumor killing activity than CAR-T produced by AAV both in vitro and in vivo.

### Methods

Primary Human T cell isolation and culture. Human T cells were isolated from healthy donors using fresh whole blood (SAILY BIO). In brief, peripheral blood mononuclear cells (PBMCs) were enriched by Ficoll (GE) density centrifugation using SepMate tube (STEMCELL). EasySep Human T Cell Isolation Kit (STEMCELL) was used to isolate CD3+ T cells from PBMC following the manufactory's instruction. Isolated T cells were activated by CD3/CD28 dynabeads (Thermo Fisher) at a bead to cell ratio of 1:3 for three days. CD3+ T cells were cultured in X-Vivo15 medium (Lonza) supplemented with 5% fetal bovine serum (FBS) (Gibco), 50 ng/ml human IL-2, 10 ng/ml IL-7 (Peprotech) and 1% Penicillin/Streptomycin. After electroporation, T cells were cultured in media with IL-2 at 100 ng/ml. Every 2-3 days after electroporation, additional media was added, along with fresh IL-2 to bring the final concentration to 100 ng/ml.

### HDR Template Production

Double-stranded DNA (dsDNA). The homologous arms and inserts of the HDR template were amplified by PCR. All fragments of HDR template were ligated by Gibson assembly (TransGen Biotech) and sequence validated. 1 pg plasmid were used as PCR template per 50 μl reaction and PCR product were column purified to prepare dsDNA.

The 5′ end modified HDR template was prepared by PCR amplification with modified primers. Primers with 5′ phosphorothioate bonds and amine C6 were ordered from Sangon Biotech (Shanghai, China). Bis-PEG10-NHS ester was purchased from Broadpharm (BP-22588) and incubated with 10 μM primers with 5′ amine C6 group and 5′ phosphorothioate bonds at 1 mM concentration in ×1 borate buffer (LEAGENE, I1H0217) overnight at room temperature. Modified primers were then desalted by concentrator tube Amicon Ultra-0.5 (Millipore). The size and purity of the amplified HDR template were confirmed by 1% agarose gel electrophoresis. PCR products were purified using DNA Clean & Concentrator PCR purification kit (ZYMO, D4034). Concentrations of HDRTs were determined by nanodrop (Thermo Fisher).

Single-stranded DNA. ssDNA was produced using Guide-it Long ssDNA Production System (Takara Bio, 632644). In general, one regular primer and a 5′-phosphorylation labeled primer was used to amplify the target DNA. Phosphorylation labeled DNA strand were further digested and the resulting ssDNA were purified and checked on agarose gel.

Single-stranded oligonucleotides (ssODN) and double-stranded oligonucleotides (dsODN). ssODN were synthesized in Sangon Biotech (Shanghai, China). dsODN is prepared by hybridizing two complementary ssODN in hybridization buffer (10 mM Tris-HCl, pH 8.0, 20 mM NaCl) and further amplified by PCR to generate large quantity.

Electroporation (EP). Three days after T-cell stimulation, cells were electroporated using 4D-nucleofector (Lonza). In general, 0.6 to 3 million cells were resuspended in 20 μl indicated EP buffer and then mixed with RNP or siRNA or DNA repair template. Cell mixture were transferred to a 20 μl electroporation cuvette and electroporated under indicated EP program. Unless indicated, electroporation was performed under P3-EO115 condition as recommended by Lonza for human T cell. After electroporation, 80 μl of pre-warmed media was added to each cuvette, and cells were allowed to rest for 10 min at 37° C. before transferring to a 96-well plate. 100 ng chemically synthesized siRNA (GenScript) were used per EP. RNPs were produced by pre-complexing Cas9 protein with sgRNA at a 1:5 molar ratio. 3 μg chemical synthesized sgRNAs (GenScript) were incubated with 3 μg Cas9-NLS (Aldevron) at 25° C. for 10 min. In HDR experiment, 0.5-3 μg donor DNA were added to the RNP complex before mixing with cells.

### Electroporation Buffer

Basic buffer A and Basic buffer B (Table 1) were prepared by admixing the respective ingredients. Basic buffer 0 is Opti-MEM medium adjusted to pH 7.2. The prepared basic buffer was filtered and sterilized with 0.2 m filter membrane. Basic buffer B or A was then mixed with opti-mem at different ratio. Five components were prepared at the following final concentration in buffer B: 0.13 mM i-inositol, 0.4 mM Ca(NO₃)₂, 0.25 mM Sodium Pyruvate, 10 mM D-glucose, 1% GlutaMAX. Mixed electroporation buffer can be stored at 4 degree for up to 6 months.

TABLE 1
Ingredients in buffer systems

| Buffer A | Buffer B | Opti-Mem (major ingredients) |
|---|---|---|
| 5 mM KCl | 5 mM KCL | 23.8 mg/L i-Inositol |
| 15 mM MgCl₂ | 15 mM MgCl₂ | 50 mg/L Ca(NO₃)₂•4H₂O |
| 90 mM NaCl | 25 mM Sodium Succinate | 27.5 mg/L Sodium Pyruvate |
| 10 mM Glucose | 25 mM Mannitol | 2.58 g/L D-glucose |
| 0.4 mM Ca(NO₃)₂ | 120 mM Na₂HPO₄/NaH₂PO₄ pH 7.2 | 332.5 mg/L L-Glutamine |
| 40 mM Na₂HPO₄/NaH₂PO₄ pH 7.2 | Adjust pH to 7.2 | 110 mM NaCl |
| Adjust pH to 7.2 | | Amino acids |
| | | Phenol red |
| | | HEPES |
| | | Serum |
| | | pH 7.0~7.4 |

Flow cytometry. Flow cytometry analysis was performed on Agilent NovoCyte Flow Cytometer. The expression of GFP and absolute cell count were performed in a 96-well flat plate. For absolute cell count, 20-50 μl cell suspension was taken and diluted 2-4 times in FACS buffer. To detect CD19BBz-CAR expression, 5×10⁵ cells were incubated with 0.5 μg of CD19-Fc (R&D Systems) in 40 μl FACS buffer (PBS+0.1% BSA+1% Penicillin/Streptomycin) for 30 min, and then stained with FITC-IgG-Fc, APC-TCRα/β antibodies (Biolegend) for 20 min on ice. The stained cells were measured on a NovoCyte flow cytometer, and analyzed with Novo Express Software. Intracellular staining was performed to detect the expression level of IFN-γ and TNF-α. AAV-CAR T and BO14-CAR T cells were co-cultured with FFluc-GFP NALM-6 at 1:1 ratio for 1 h. 2 μM Monensin (Biolegend) was added to the co-culture for another 5 hours before staining. CAR staining is performed first, followed by fixation and permeabilization. Antibodies against IFN-γ and TNF-α were incubated for 30 min and cells were analyzed by flow cytometer.

### Subcellular Analysis of DNA Distribution in Primary T Cells

dsDNA labeling 20 μg of dsDNA (1350 bp) was incubated with 10 l Reagent of Cy5 Label-IT kit for 1 hour at 37° C. (Mirus Bio) prior to column purification (ZYMO RESEARCH). The purified DNA was quantified with NanoDrop and stored away from light at −20° C. 1×10⁶ human primary T cells were transfected with 1 μg labeled dsDNA, and cells were collected 10 min post-EP for downstream analysis.

Staining and confocal analysis. Cells were fixed in 4% formaldehyde for 10 min at room temperature and were incubated with Phalloidin. Stained cells were resuspended to 50-100 μl with PBS and cytospinned on microscope slides for 10 min at 500 g. After the spin, 1 drop of DAPI-mounting medium (Sigma, F6057) was added on the slide and covered with a coverslip. Cy5-labeled dsDNA was localized and photographed using Zeiss LSM880 confocal microscope under a 60× oil objective. The field of vision was randomly selected and the focal length was adjusted based on Cy5 fluorescence. The DAPI was used to visualize the cell nuclei, phalloidin to estimate the cell borders and Cy5 is used to trace DNA distribution. Nuclear localization ratio was obtained by manual count. The mean fluorescence intensity of Cy5-labeled dsDNA within the nucleus and the entire cell were calculated using the image analysis tools of the Zeiss ZEN 2.3 Image Processing Software.

### Quantitative Real-Time PCR

4 hours post-EP, 5×10⁵ cells were collected for RNA extraction (Magen, #R4122-03) and cDNA preparation using HiScript II Q Select RT SuperMix (Vazyme, #R223-01). Real-time PCR reactions were performed with ChamQ Universal SYBR qPCR Master Mix (Vazyme #Q711-02) on a Bio-Rad CFX Connect 96-well based on the manufacture instructions. The expression level of each indicated gene was normalized to GAPDH.

### Cell Lines and Culture Conditions

Nalm6 cells and THP1 cells (BNBIO, China) were cultured in RPMI1640 (Gibco) medium with 10% FBS (AusGeneX) and 1% Penicillin/Streptomycin (Gibco). HEK293T17 (ATCC) cells cultured in DMEM (Gibco) medium with 10% FBS and 1% Penicillin/Streptomycin.

Nalm6-luciferase-GFP cell line (FFluc-GFP Nalm6-CD19⁺) was generated by infecting Nalm6 with lentivirus carrying a GFP-P2A-luciferase construct. GFP expressing cells were sorted and expanded for further experiments. Nalm6-luciferase-GFP-CD19 knockout cell line (FFluc-GFP Nalm6-CD19⁻) were generated by knocking out CD19 via a lentivirus carrying sgRNA against CD19 and spCas9. CD19 negative cells were FACS sorted out to establish the control cell line. DNA analysis by TIDE were performed to confirm the genetic ablation of CD19.

### Western Blot Analysis

5×10⁵-1×10⁶ cells were lysed by ice-cold RIPA buffer (Beyotime) containing protease inhibitors (Beyotime) and incubated on ice for 15 min. Protein supernatant was collected after centrifuging at 13,000 g 4° C. for 5 min. Protein concentration was determined using the BCA Protein Assay Kit (Beyotime). Proteins were separated by 10% SDS-PAGE, followed by immunoblot analysis with the indicated antibodies.

### Generation of AAV1 Transduced CAR T Cells

AAV1 was produced by triple transfection of AAV2/CD19BBz, AAV1 serotype plasmid, and a helper plasmid via polyethyleneimine (PEI) in HEK293FT cells. Both cell supernatant and cell pellet were collected at 72 h for AAV purification. AAV is purified using iodixanol density gradient. In brief, cell pellet was lysed by repeated freeze-thaw cycles to release viruses, followed by benzonase (Sigma) treatment to remove gDNA and RNA. To collect AAV from supernatant, 0.0233 g/ml NaCl and 0.085 g/ml Polyethylene Glycol 8000 were added sequentially. The mixture was centrifuged at 6600 rpm at 4° C. for 15 min and the pellet was suspended in 1×GB buffer (0.5M NaCl, 0.1M MgCl₂, 0.1M Tris pH 7.6). Resuspended pellets containing AAVs were added to a discontinuous iodixanol gradients and centrifuged at 48000 rpm for 2 h. AAVs located at 40-60% layer was extracted via a syringe and followed by concentration via Millipore Amicon filter unit (Millipore). Virus titer was determined by qPCR using ChamQ Universal SYBR qPCR Master Mix targeted to CAR. To generate AAV-CAR, stimulated T cells were first electroporated with RNP. 10 min post-EP, AAV1-CAR were added at a MOI=5×10⁵. Cells were washed 1-2 h post AAV1 infection.

### In Vitro Killing Assay

FFluc-GFP Nalm6-CD19+ was used as target antigen cells, and FFluc-GFP Nalm6-CD19⁻ was used as control cells. The effector (AAV-CAR; BO14-CAR) and target cells or control cells were co-cultured in triplicates at indicated E/T ratios using 96 U-bottom well plate with 1×10⁴ target cells or control cells in a total volume of 150 μl per well in Nalm-6 culture medium. 18 h later, luciferase assay intensity was measured by a plate reader (MD SpectraMax i3×) with Luciferase Assay System (Promega). The number of tumor cells was calculated according to the curvature.

### In Vivo Mouse Systemic Tumor Model

6- to 8-week-old NOD/SCID/IL-2Rγ null mice (Gempharmatech, China) were inoculated with 2×10⁵ FFluc-GFP Naml6 cells by tail vein injection. 4 days post-tumor cell injection, AAV-CAR or BO14-CAR T cells were injected at a dose of 5×10⁵ per animal. Prior to imaging, 4.5 mg D-Luciferin potassium (Meilunbio, China) was intraperitoneal injected into each animal. Bioluminescence imaging were performed under the SI Imaging AmiX Imaging System (Spectral Instruments Imaging, USA) with Living Image software (Spectral Instruments Imaging) for acquisition of imaging datasets. All relevant animal-use guidelines and ethical regulations were followed.

### Results

Non-viral reprogramming of primary human T cells provides a safe, fast and virus-free generation of CAR-T cells. Unlike lentivirus-mediated random integration or site-specific integration of CAR construct using AAV as templates, non-viral reprogramming of CAR-T cells circumvents the need of lentivirus or AAV preparation for donor DNA delivery and can be quickly adopted for manufactory process development. However, the biggest hurdle that holds back the non-viral delivery is the cellular toxicity after the electroporation of DNA donor and the relative low delivery efficiency into human T cells.

To address this issue, we first explored the mechanism of DNA electroporation induced T cell toxicity. In healthy cells, DNA is restricted to the mitochondria and nucleus. Cyclic GMP-AMP synthases (cGAS) detects cytoplasmic DNA and signals through stimulator of interferon gene (STING) to initiate an innate immune response. We hypothesized that cGAS-STING was essential to trigger electroporation of DNA-induced cellular toxicity in human T cells.

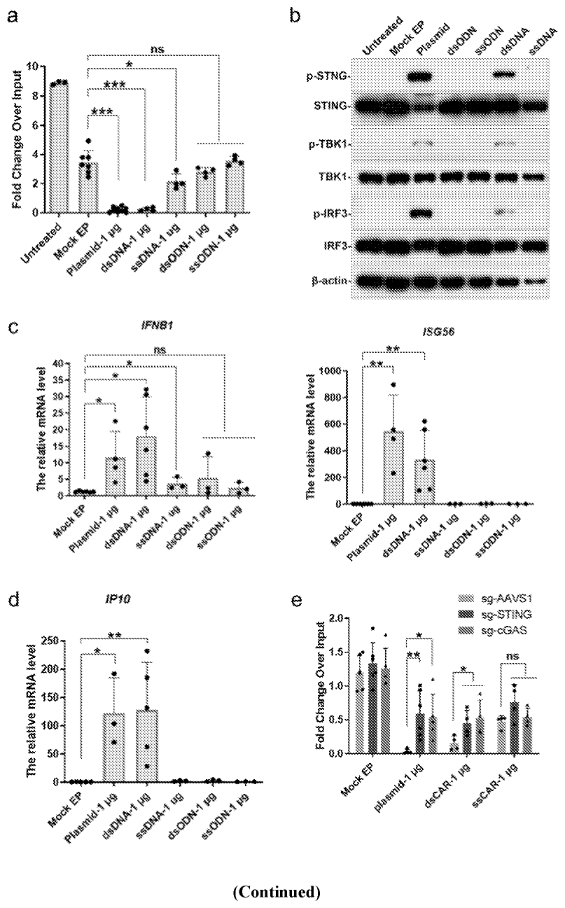

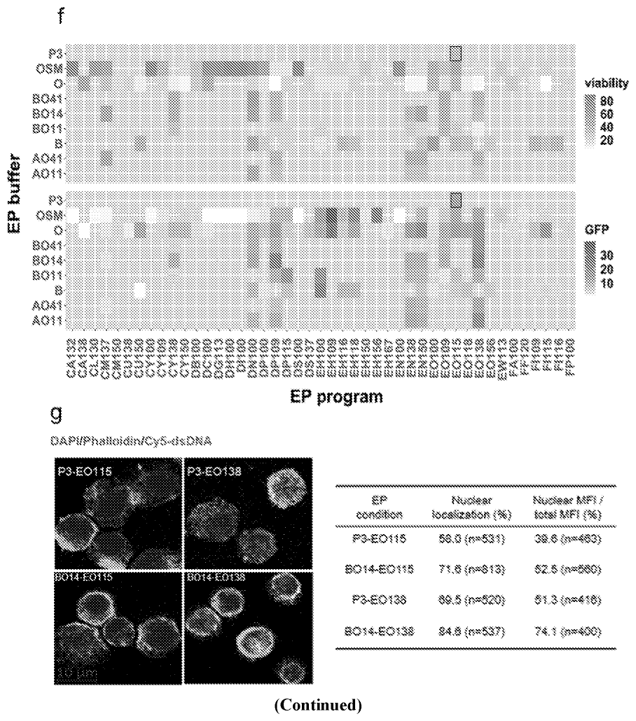

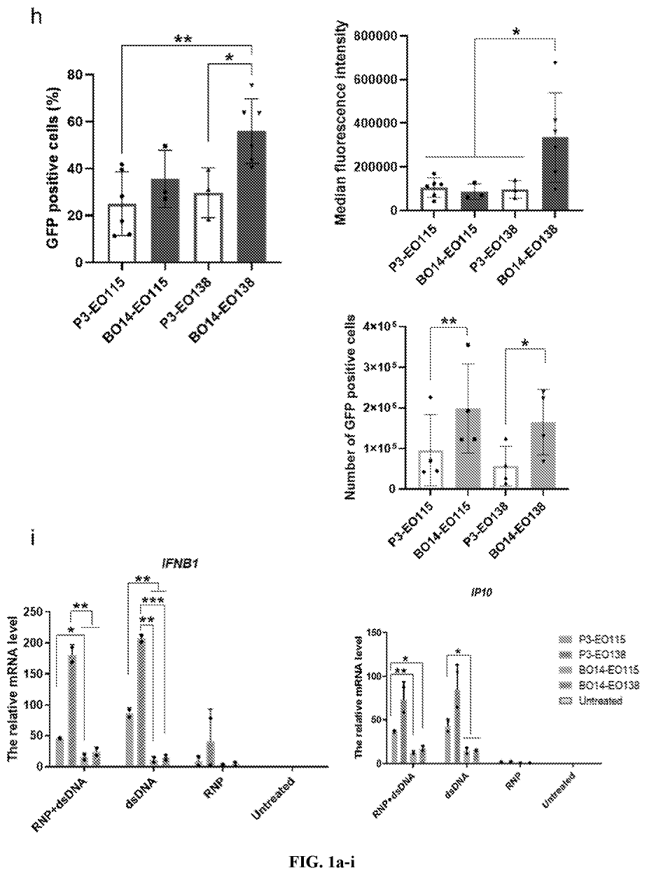

To examine this hypothesis, we first determined which types of DNA was most detrimental to primary human T cells. Five types of DNA including a 3.5 kb plasmid, a 1.4 kb linear double-stranded DNA (dsDNA), a 1.4 kb single-stranded (ssDNA), a 99-mer double-stranded oligonucleotides (dsODN) and a 99-mer single-stranded oligonucleotide (ssODN) were electroporated into T cells and cell numbers were counted. dsDNA and plasmid showed ˜14-17 fold loss of cells when compared with MOCK electroporation (MOCK EP) (FIG. 1a). In contrast, ssDNA, which shared the same sequence as dsDNA, only triggered a minor cell loss (1.6 fold). Shorter sequences such as dsODN or ssODN did not induce significant cell loss (FIG. 1a).

To determine whether cell loss is a result from cGAS-STING activation, we examined STING phosphorylation (p-STING) and its downstream signaling pathways phosp-TBK1 (p-TBK1) and phosp-IRF3 (p-IRF3). Similar to MOCK EP control, dsODN, ssODN and ssDNA did not exhibit any activation, whereas plasmid and dsDNA transfected T cells showed strong activation of p-STING and p-TBK1 and p-IRF3 (FIG. 1b). One signature of STING activation is its downstream production of inflammatory cytokines and type I interferon (IFN) gene expression. In support of western blot results, IFNβ1, IFN-stimulated gene 56 (ISG56), and Interferon gamma-induced protein 10 (IP10), the signature reporters of type I IFN and inflammatory cytokines respectively, were upregulated in cells electroporated with plasmid or dsDNA (FIG. 1c-d).

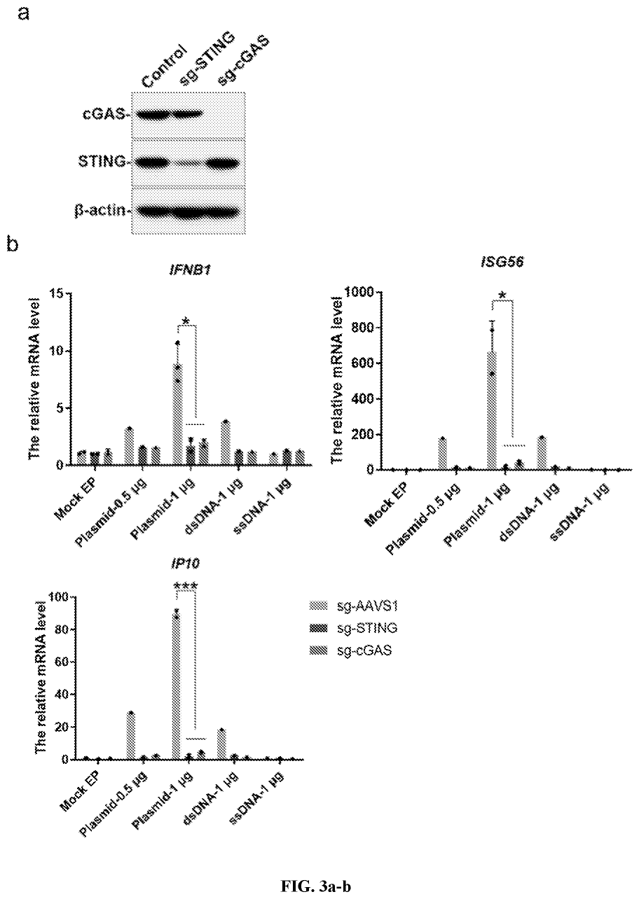

We next sought to ask if DNA induced T cell toxicity can be rescued by genetic ablation of cGAS-STING mediated immune response pathway. sgRNAs against cGAS or STING were screened and used to effectively knockout cGAS and STING expression prior to DNA electroporation (FIG. 1e). Compared to a mismatch gRNA control targeting AAVS1, cGAS or STING knockout cells exhibited 3 fold to 16 fold increase of cell number when challenged with dsDNA or plasmid respectively (FIG. 1e). Analysis of STING downstream inflammation and type I interferon pathway showed dramatic decrease in the activation of signature genes upon DNA electroporation in cGAS or STING knockout cells (FIG. 3b).

To test whether this is the case in mouse, we isolated T cells from cgas or sting null mice and electroporated with 1 μg plasmid. Similar to human T cells, mouse cgas^{-/-} or sting^{-/-} T cells showed 17.5 fold more viable cells than wild-type, and significantly decreased cGAS-STING downstream gene expressions. To confirm that STING is the key player, we restored full-length or STING bearing a C88/91A or C88/91S mutation, a well-characterized palmitoylation site. T cells isolated from sting^{-/-} mouse were infected with retrovirus and followed by plasmid challenge. Restoring full-length Sting induced significant cell death and upregulation of signature genes compared vector control. Interestingly, STING bearing C88/91A or C88/91S mutation expression still showed significant cell death, indicating that STING palmitoylation is not essential for T cell death. Taken together, these data suggested that cGAS-STING activation is the major cause of dsDNA electroporation-induced cell loss in primary human T cell.

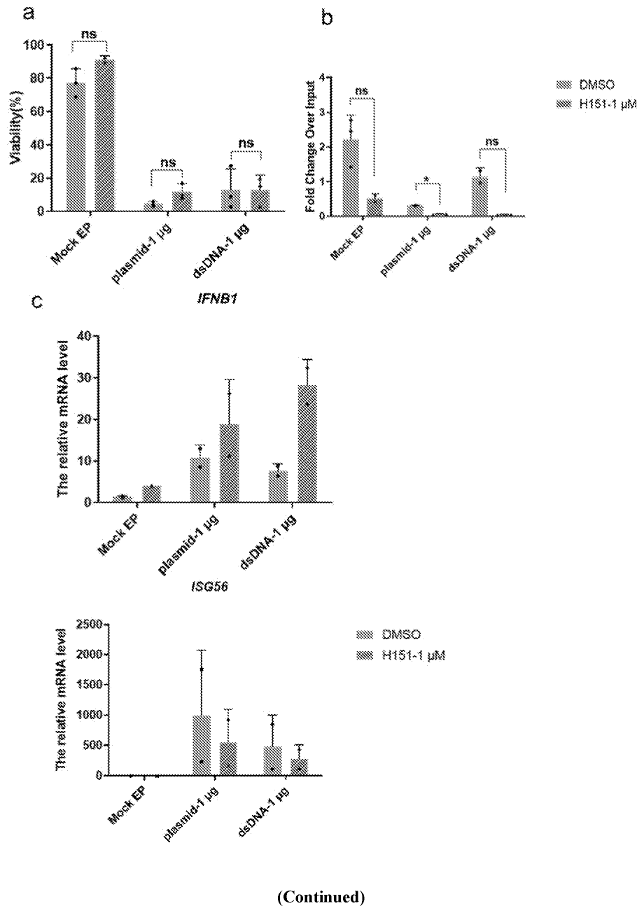

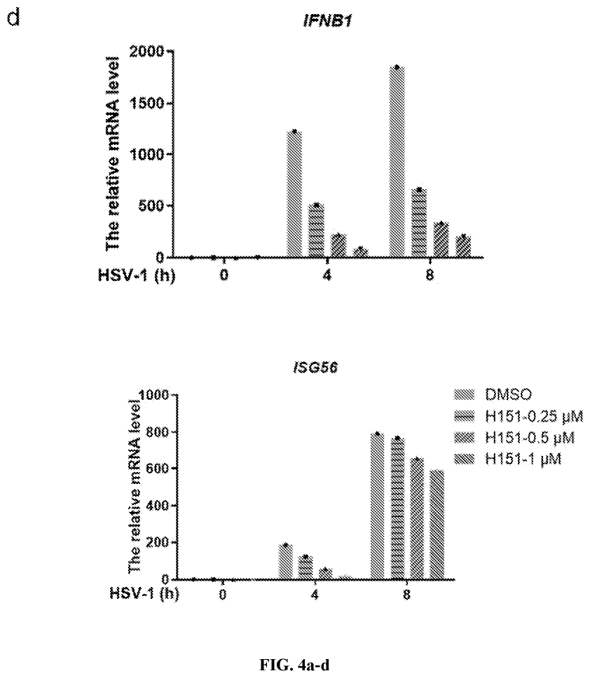

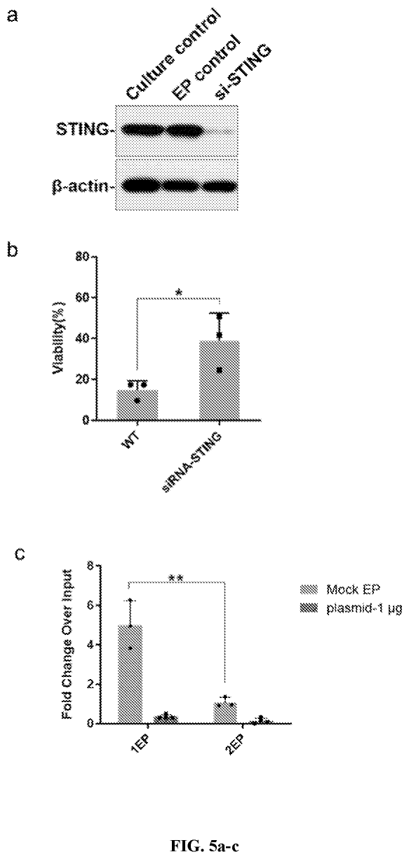

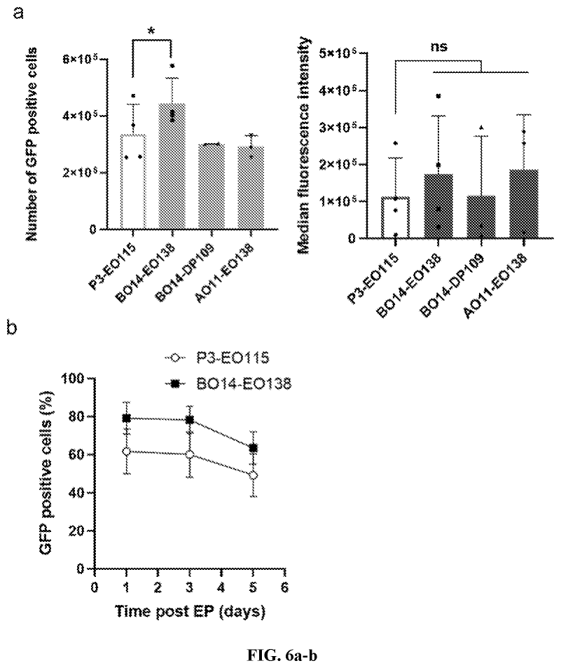

cGAS-STING pathway is crucial for innate immune response and other biological functions. We next sought to transiently inhibit this pathway by small molecule inhibitor H151 or siRNA-mediated knockdown. Unfortunately, H151 treatment failed to rescue DNA-induced cell death and in align with this, IFNβ and ISG56 were upregulated in DNA transfected T cells (FIG. 4a-c). It is noted that H151-treated THP1 cells showed positive dose-response when challenged with herpes simplex virus type 1 (HSV-1), a DNA virus, suggesting that H151 or cGAS-STING may function differently in T cells (FIG. 4d). Indeed, H151 is known to function to block STING palmitoylation, which is important in initiating interferon-dependent signaling activation in innate immune cells such as macrophages. However, in T cells, STING palmitoylation is not essential for STING-related cell death. We then used siRNA to transiently knockdown STING (FIG. 5a). STING attenuated T cells showed increased cell viability post DNA electroporation (FIG. 5b). Though siRNA strategy can rescue DNA induced cell loss, the introduction of a second electroporation to deliver siRNA was not well tolerated, as evidenced by little cell growth post-2^{nd} electroporation even in MOCK EP control (FIG. 5c).

cGAS is a cytosolic DNA sensor and does not react with nucleus DNA. Therefore, another strategy is to promote nuclear delivery of DNA, and thereby bypassing the surveillance of cytosolic cGAS sensor. To achieve this, we performed high throughput screening of different electroporation buffer and program combinations that enable nuclear delivery of DNA in primary human T cell (FIG. 1f). Using transfection efficiency and cell growth as a surrogate, we identified several buffer/program combinations with enhanced plasmid delivery than the commercial recommended Lonza P3 buffer and E0115 program (FIG. 6a-b).

Of three electroporation conditions, buffer BO14 paired with E0138 program had the highest number of GFP expressing cells, much higher than the commercial buffer (FIG. 6a-b). We then measured the cellular distribution of a Cy-5 labeled 1.35 kb dsDNA right after electroporation. Cells were electroporated under either BO14 buffer or P3 buffer with indicated EP programs and then stained with Phalloidin and DAPI to outline cytoplasm and nuclei. Quantification of more than 400 cells showed that buffer BO14 has 84.6% nuclear delivery efficiency compared with 69.5% under P3-based delivery. In addition, the ratio of nucleus florescence intensity was also higher when delivered via BO14 conditions, as compared to P3 commercial under the same EP programs, 74.1% vs 51.3% and 52.5% vs 39.6% for E0138 program and EO115 program, respectively (FIG. 1g).

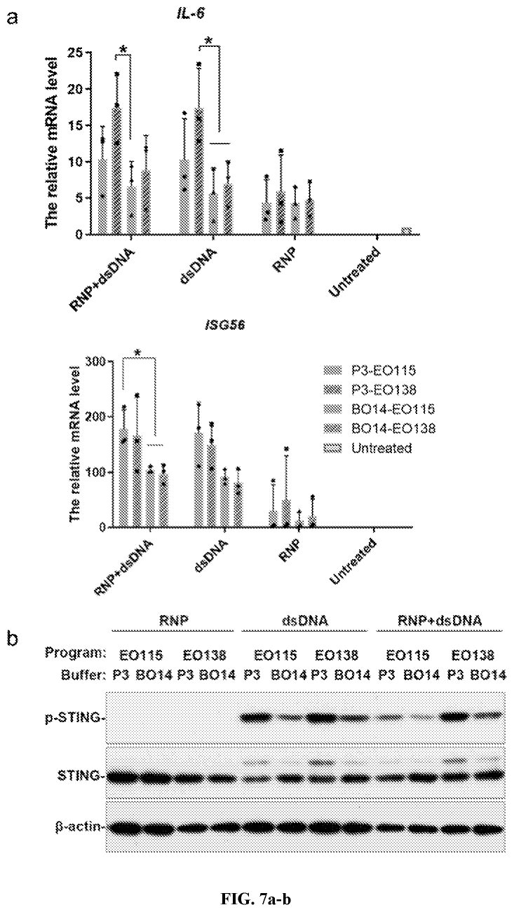

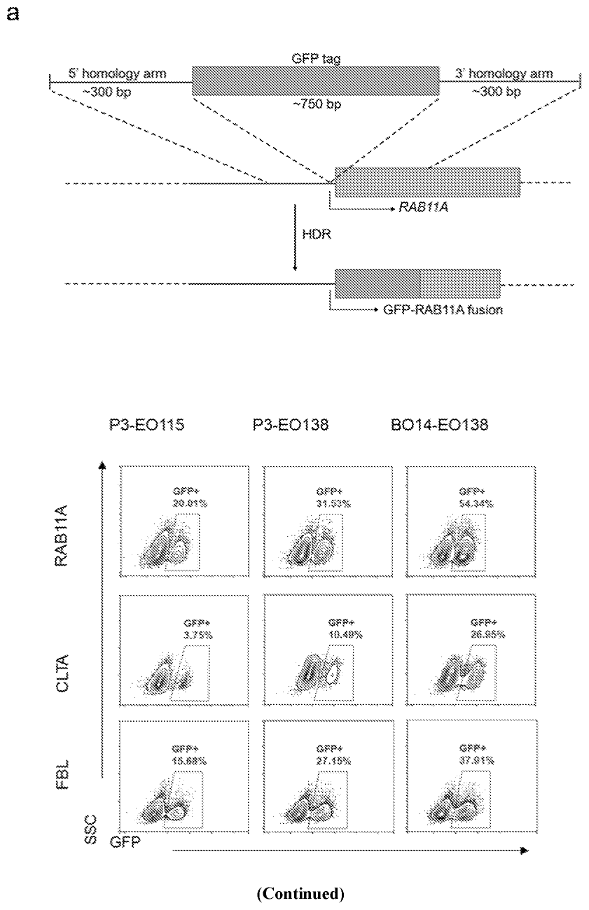

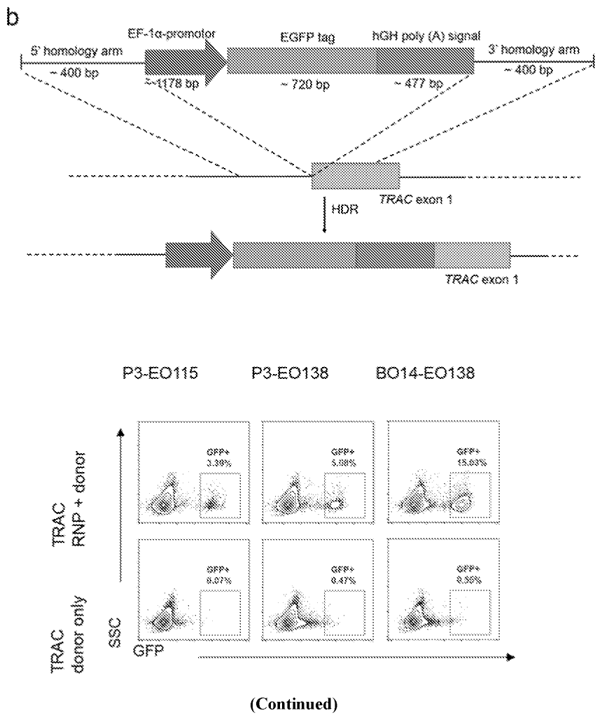

In alignment with enhanced nuclear delivery, cells electroporated with dsDNA encoding an EF1α promoter followed by EGFP exhibited enhanced GFP cells number and expression level under BO14-mediated delivery compared with P3 commercial buffer (FIG. 1h). To determine whether BO14 buffer-facilitated nucleus delivery could mitigate cGAS-STING activation, we examined cGAS-STING downstream pathway. Both STING phosphorylation level and its downstream reporter genes IFN-0, ISG56, and IP10 were downregulated (FIG. 1i and FIG. 7a-b), demonstrating that enhanced nuclear DNA delivery mitigated cGAS-STING immune response pathway and thus promoted T cell survival.

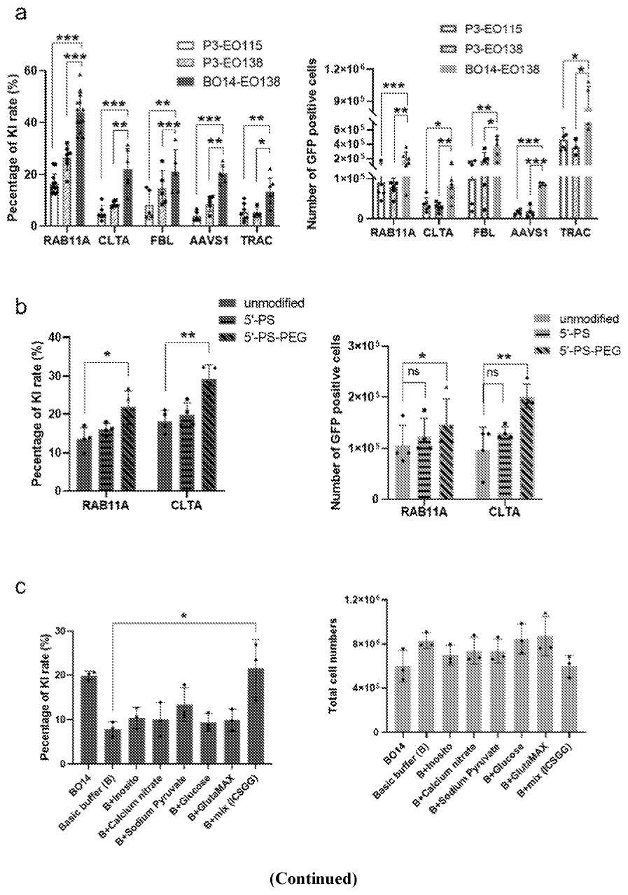

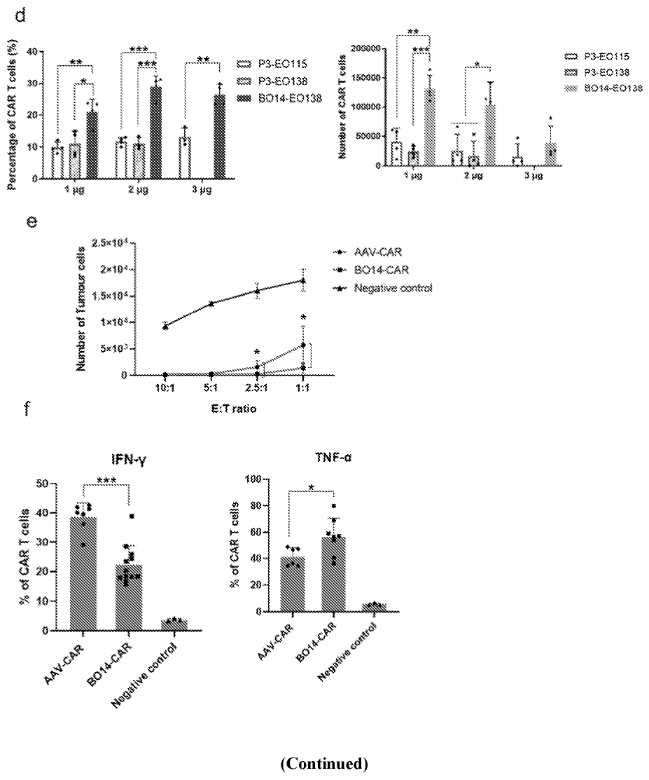

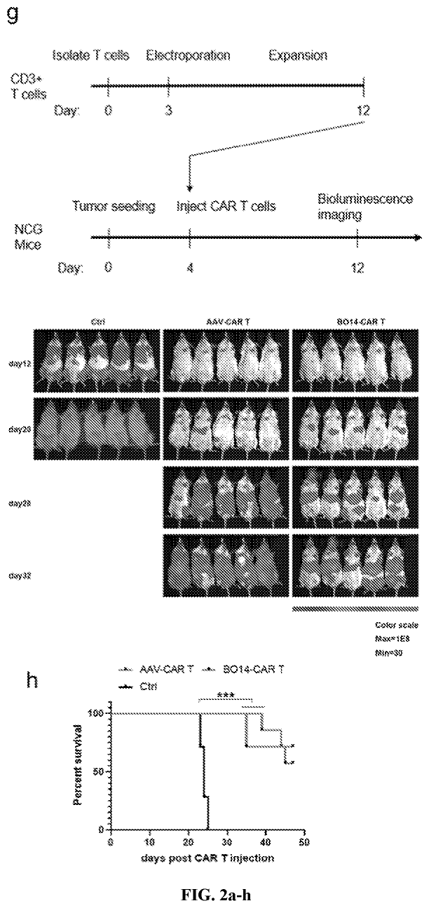

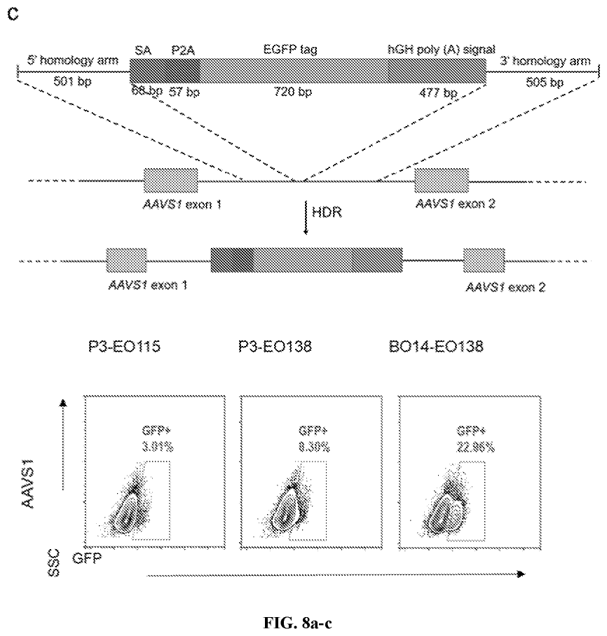

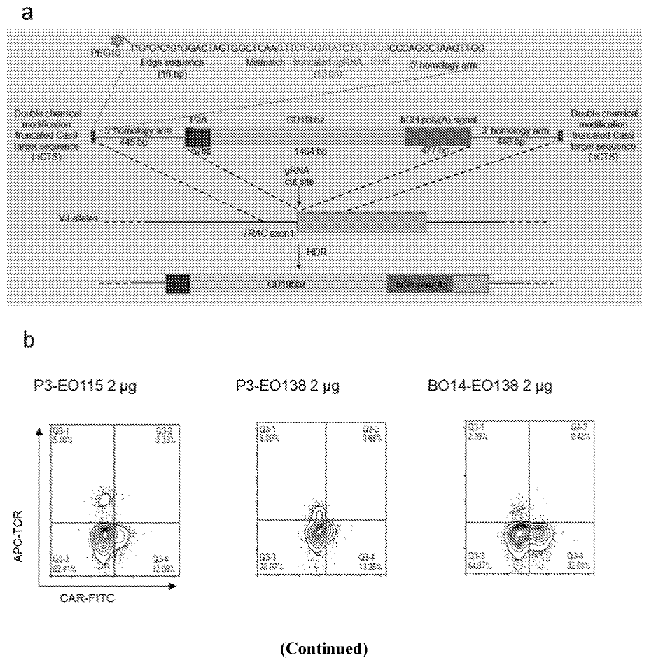

Nucleus delivery of DNA donor is critical for homologous directed repair (HDR). We next tested whether the system could improve CRISPR/Cas9 mediated target insertion. We chose five different loci and tested three knock-in strategies including targeting exons or introns, or inserting an EF1a driven EGFP construct (FIG. 8a-c). Across five different loci tested, BO14-based non-viral delivery showed about 2-5 fold increase in absolute targeted knockin cells compared with commercial P3 based dsDNA template delivery (FIG. 2a and FIG. 8a-c). Previous studies have shown that donor template modification could greatly enhance HDR efficiency. To determine whether our system is compatible with DNA donor modification, we synthesized 5′ phosphorothioate (PS)- or 5′ PS and C6-polyethylene glycol 10 (PEG) double modified primers and applied modified primers to generate 5′ modified DNA donor. We observed a moderate but statistically significant increase of knock-in rate and knock-in cells when donor is modified with 5′ PS and PEG (FIG. 2b), suggesting that the BO14-based delivery is compatible for donor DNA modification with no obvious cellular toxicity.

BO14 buffer is composed of basic buffer B mixed with opti-mem at 1:4 ratio. To determine the key components that are crucial in mediating nuclear delivery, we subtracted potential important components in opti-mem and added back to basic buffer B one by one. While each component showed a minor increase of knock-in rate compared with basic buffer alone, the mixture of all five components (Inositol, Calcium nitrate, Sodium pyruvate, Glucose, GlutaMAX, collectively “ICSGG”) showed a comparable knock-in rate and cell number to BO14 buffer (FIG. 2c). Accordingly, we have now a defined composition of B+ICSGG electroporation buffer, and a similar B+2xICSGG electroporation buffer (Table 2).

TABLE 2
Major ingredients in combination buffers

| Ingredients | BO14 | | BO11 | | B-ICSGG | | B-2xICSGG | |
|---|---|---|---|---|---|---|---|---|
| i-Inositol | 19.04 | mg/L | 11.9 | mg/L | 23.8 | mg/L | 47.6 | mg/L |
| Ca(NO₃)₂•4H₂O | 40 | mg/L | 25 | mg/L | 100 | mg/L | 200 | mg/L |
| Sodium Pyruvate | 22 | mg/L | 13.75 | mg/L | 27.5 | mg/L | 55 | mg/L |
| D-Glucose | 2060.4 | g/L | 1287.75 | g/L | 1801 | mg/L | 3602 | g/L |

| | | | | | | |
|---|---|---|---|---|---|---|
| L-Glutamine | 266 | mg/L | 166 | mg/L | 0 | 0 |

| | | | | | | |
|---|---|---|---|---|---|---|
| GlutaMAX | 0 | 0 | 434 | mg/L | 868 | mg/L |

| | | | | | | |
|---|---|---|---|---|---|---|
| NaCl | 5198.38 | mg/L | 3248.99 | mg/L | 0 | 0 |

Chimeric Antigen Receptor (CAR) reprogrammed T cells are a powerful immunotherapy option for several hematological malignancies. Recent study of CAR-T cell reprogramming has found out site-specific insertion of CARs into endogenous TRAC locus using CRISPR/Cas9 and an adeno-associated virus vector to deliver the donor template has improved functionality over the conventional lentivirus transduced counterparts.

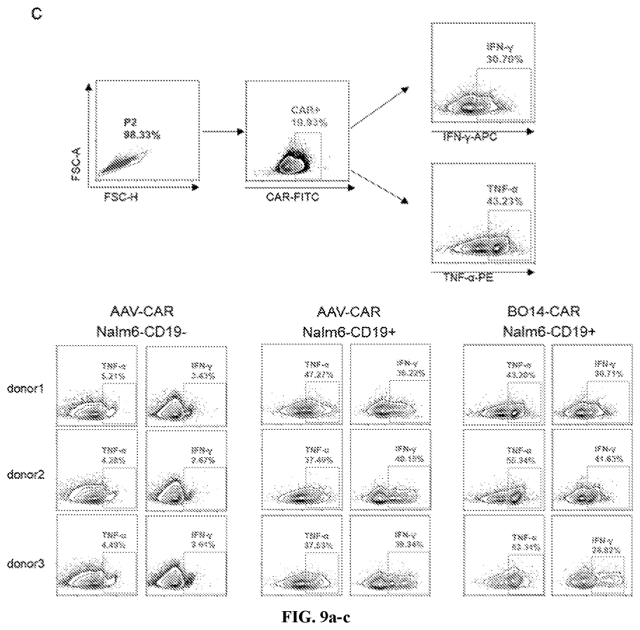

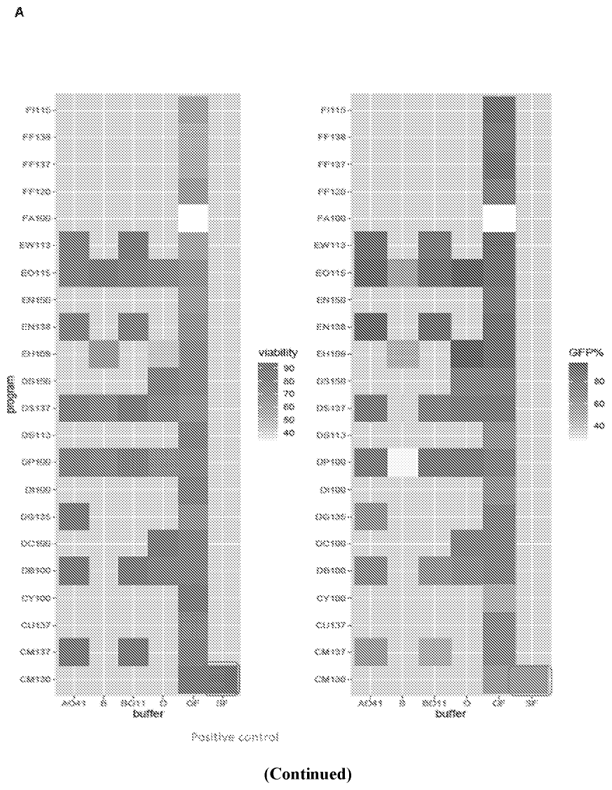

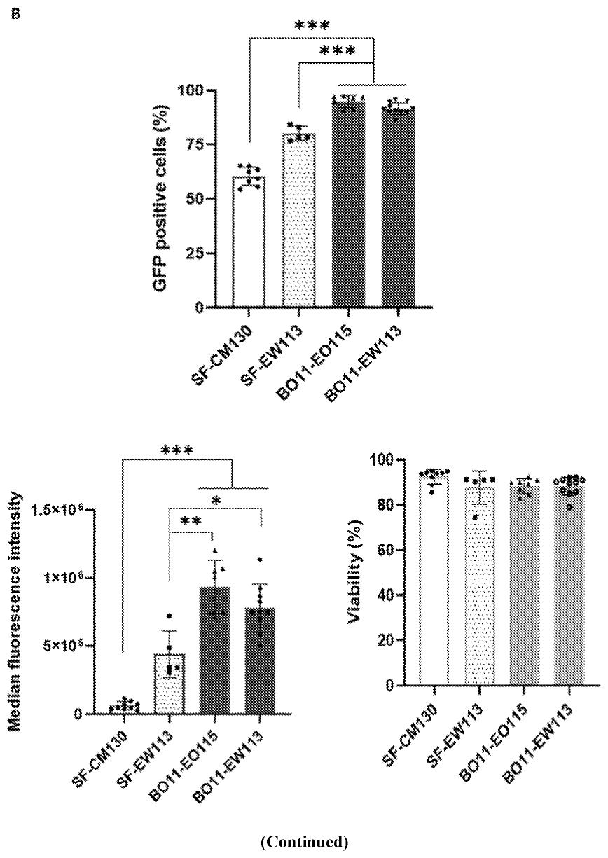

To expand the usage of our system, we inserted the CD19-specific 19BBz CAR construct into TRAC locus so that the expression of CAR is under the endogenous promoter (FIG. 9a). Specifically, a 3 kb dsDNA were co-electroporated with Cas9 RNP complex targeting TRAC locus to generate site-specific integration of CD19BBz CAR via homologous recombination. We observed up to 32% CAR-T insertion via BO14-mediated delivery compared with 12% via P3 buffer (FIG. 2d and FIG. 9b), and up to 47% CAR-T insertion via B+ICSGG-mediated delivery. The absolute CAR-T cells generated by BO14-mediated delivery also outnumbered the P3-mediated delivery by 2-5 fold, indicating reduced toxicity by BO14 (FIG. 2d), or up to 21 fold with B+ICSGG-mediated delivery. Also noted, the amount of DNA template used in this study is 1-2 μg/electroporation, about 4-5 fold lower than previous reports. This could greatly ease the manufactory process as low dose of DNA donor could still achieve efficiency CAR-T cells. To test whether the new buffer may affect T cell fitness, we compared the proliferative capacity, the ability to secrete cytokines (IFN-γ, IL9, TNFα) and exhaustion markers (PD1, LAG3, TIM3, and CD154) of human T cells that were electroporated in P3 or B+ICSGG buffer. We followed T cell proliferation over 7 days post MOCK electroporation under EO115 or EO138 program and found similar viability and cell growth. qPCR analysis of exhaustion markers and cytokines at 6 hour or 24 hour post-electroporation did not identify any difference between buffers. Together, these data showed that the new buffer-mediated electroporation was able to generate efficient CAR-T cells without any obvious effect on T cell fitness.

Next, to determine whether BO14-mediated target insertion of CAR-T is functional, we compared non-viral based methods with AAV-mediated donor delivery. In both cases, CAR sequence and RNP are the same to follow the same integration strategy. The only difference is the method of donor template delivery. In non-viral based methods, 5′ PS-PEG modified dsDNA were co-electroporated with RNP whereas in AAV-mediated delivery, RNP were electroporated first, followed by AAV addition. Equal amount CAR-T cells were co-cultured with tumor cells expressing CD19 antigen Nalm6-luciferase cells or Nalm6-CD19 KO cells. When CAR-T were incubated with tumor cells as 5:1 ratio, all tumor cells were lysed by non-viral delivered CAR or AAV-CAR; when incubated at 1:1 ratio, five-fold lower tumor cells were left in BO14 CAR-T, indicating that BO14 generated CAR-T has equal or higher tumor killing potency than AAV-CAR (FIG. 2e).

Likewise, the B+ICSGG buffer was tested for non-viral delivery, as compared to AAV- or lentivirus-mediated delivery. In lentivirus-based delivery, the same CAR sequence was used and purified lentivirus (MOI=10) was used to infect human T cells. Compared with lenti-CAR, AAV-CAR or P3-CAR, B+ICSGG-CAR exhibited the highest CAR expression intensity among all methods and significant enhanced CAR-T positive percentage than lentiviral or P3 based methods. Long term track of CAR-T production also showed that the absolute B+ICSGG-CAR T outnumbered that of lentivirus.

The higher lysis efficiency of non-viral delivered CAR likely resulted from the higher secretion of TNF-α, the anti-tumor cytokines (FIG. 2f and FIG. 9c). Finally, we confirmed that BO14-generated CAR-T cells have in vivo anti-tumor function by adoptive transfer of equal amount of BO14-CAR T or AAV-CAR T cells into NCG mouse bearing Nalm6-tumor cells. Twelve days post-CAR T injection, mouse injected with BO14-CAR T cells exhibited a 1.6-fold decrease of tumor burden than AAV-CAR treated animal, consisting with in vitro killing result (FIG. 2g), and prolonged survival (FIG. 2h). Taken together, these data demonstrate that non-viral nucleus-targeting DNA delivery could generate large amount and high-quality CAR-T cells.

In summary, this example described a simple and effective strategy for non-viral genome engineering by mitigating cGAS-STING activation through improved nuclear delivery of dsDNA. Evaluation of five different repair template DNA formats, we found plasmid and dsDNA induced the most profound loss of primary T cells and cGAS-STING activation compared with ssDNA, dsODN or ssODN (FIG. 1a-d). Genetically intervention of cGAS or STING could rescue cell death phenotype and render tolerance to DNA stimulation, suggesting attenuation of cGAS-STING activation is the key solution for effective non-viral genome editing (FIG. 1e). Although ssDNA only triggered a minor immune response and cell death, ssDNA is likely to be mutated by the endogenous deaminase and it is difficult to manufacture. Thus, ssDNA may not be a suitable donor format for CAR-T engineering.

To mitigate STING activation, we systematically optimized electroporation system and identified a new buffer BO14 that showed enhanced nuclear delivery of dsDNA with reduced cGAS-STING activation. The new methods exhibited 2-5 fold increase in targeted insertion cells across multiple loci in primary human T cells (FIG. 2a). BO14-engineered CAR T cells exhibited up to 2-fold increase of insertion rate and the absolute CAR-T cells number is 5 fold higher than the commercial based non-viral system (FIG. 2d). Also noted, BO14-CAR T cells exhibited enhanced tumor killing activity than CAR T cells produced via AAV-based donor delivery, likely due to higher TNF-α secretion from BO-14 CAR T cells (FIG. 2e-f).

The present method has several advantages in combination (FIG. 15): 1) enhanced KI efficiency, 2) increase of total knock-in cells by reducing damage to cells, thereby no requirement of further cell enrichment, 3) higher tumor cell killing activity and 4) short workflow to be quickly adapted to manufacturing process for clinical use. The discovery that buffer optimization could enhance targeted insertion identify a new direction of improving non-viral mediated genome editing.

### Example 2: Testing of Gene Delivery in Other Cell Types

This example tested the use of the developed buffer and a variant for nucleus-targeted DNA delivery in various types of cells.

The new electroporation (EP) protocols, e.g., BO buffers used with corresponding programs, were tested for plasmid and RNP delivery into 293T cells. As noted in Example 1, the BO14 buffer contained basic buffer B mixed with opti-mem at 1:4 ratio. Likewise, the BO11 buffer contained basic buffer B mixed with opti-mem at 1:1 ratio.

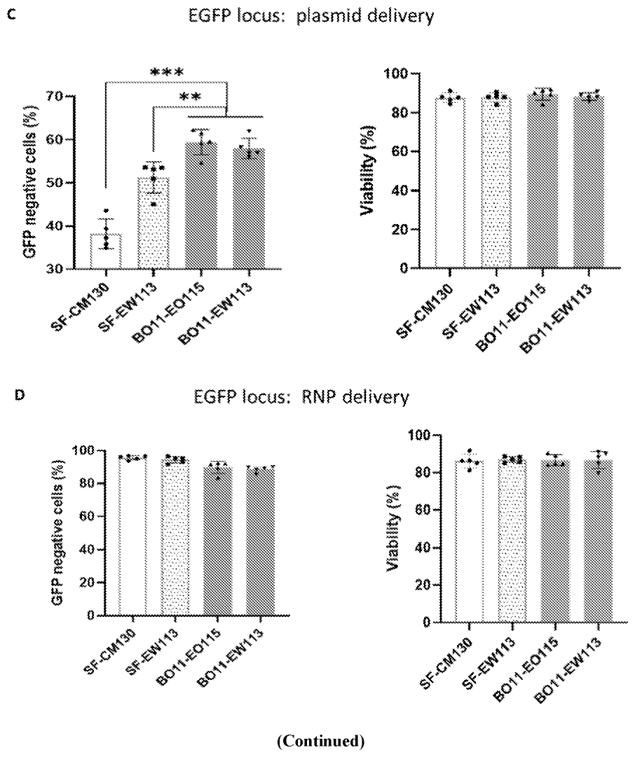

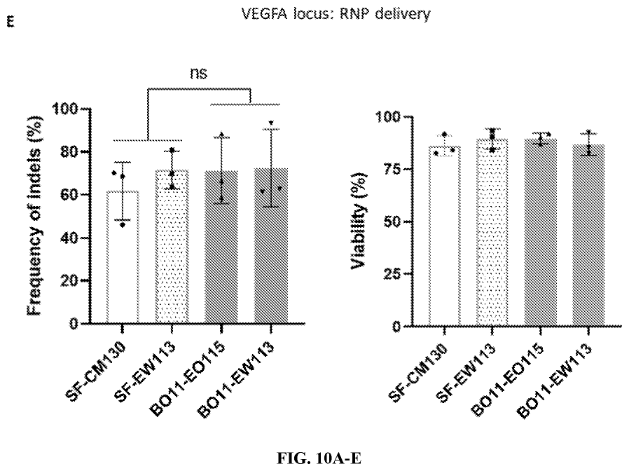

As shown in FIG. 10A-F, BO11, with both E0115 and EW113 programs, greatly enhanced gene delivery in 293T cells, as compared to commercial Lonza buffers (SF).

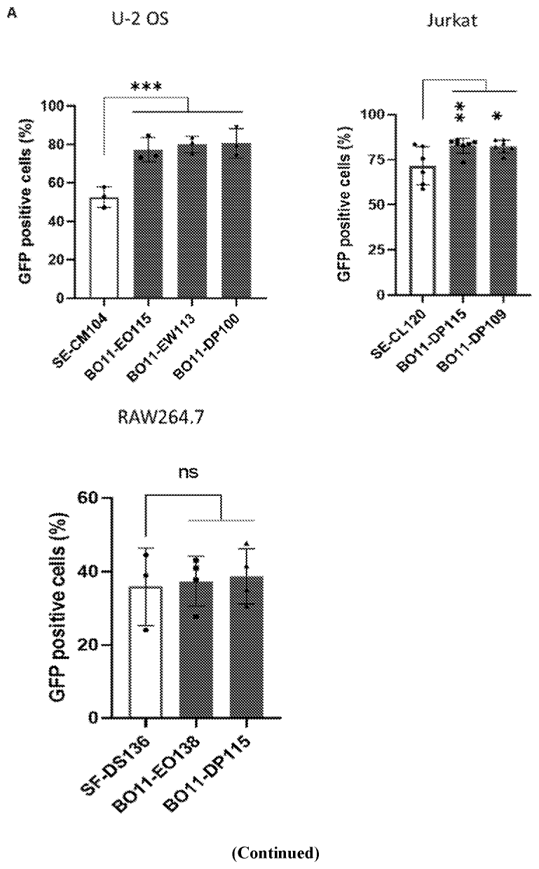

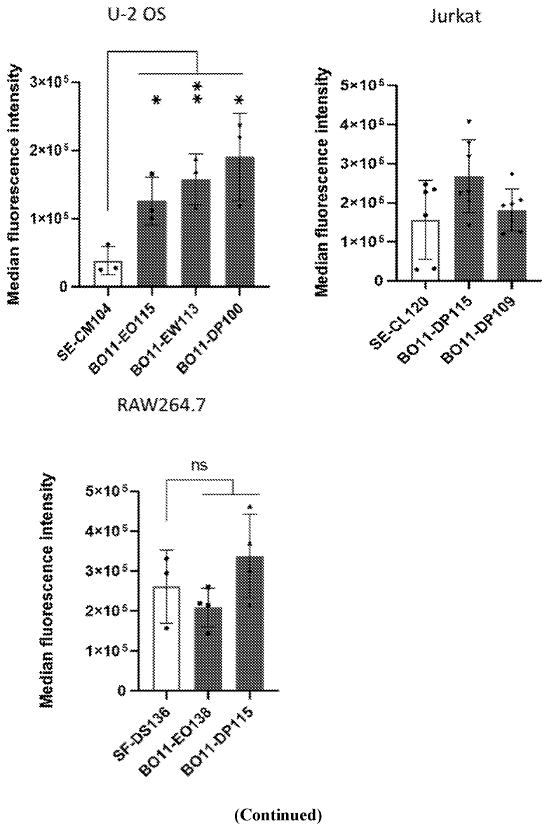

Similarly, the new EP protocol with the BO11 buffer was used for gene delivery to U-2 OS, jurkat, and Raw264.7 cell lines. The results are presented in FIG. 11A-C, which show that the EP protocol resulted in higher efficiency in delivery as compared to the commercial buffers.

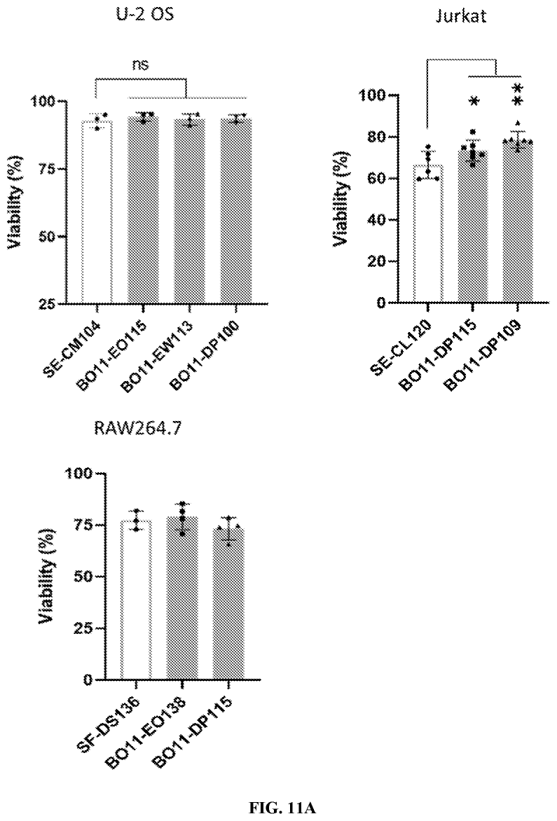

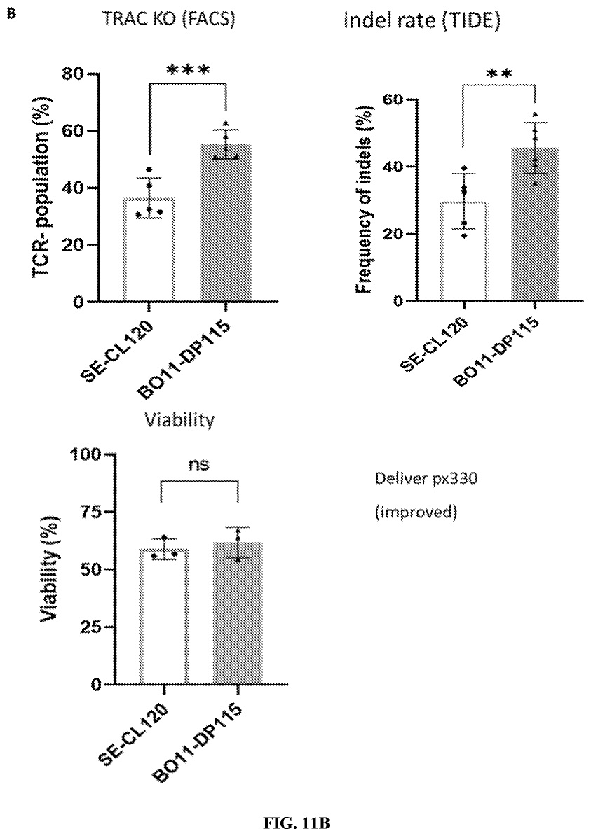

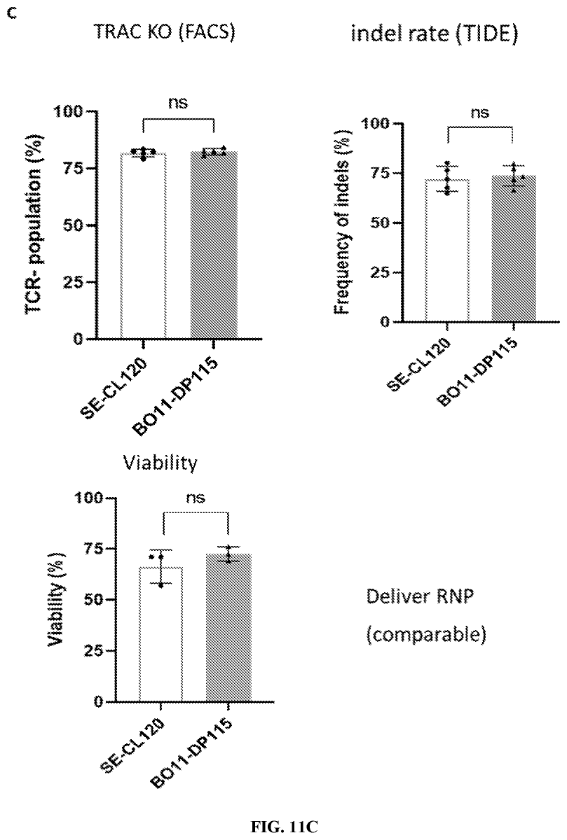

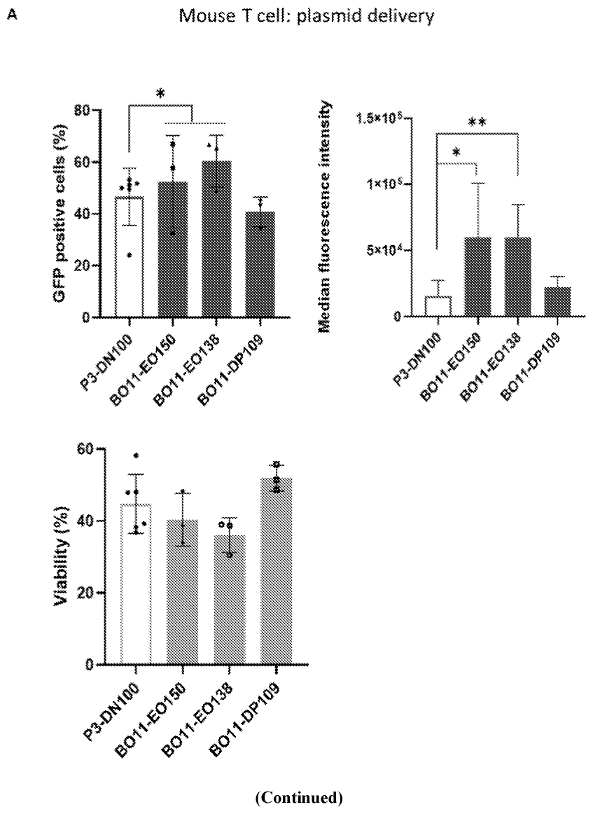

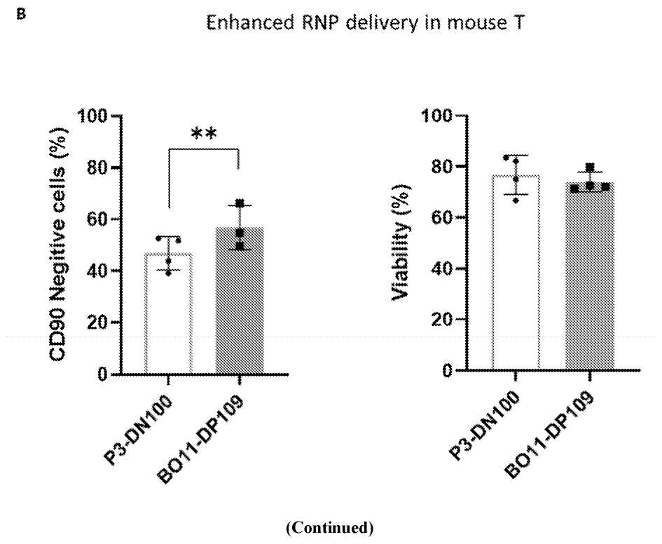

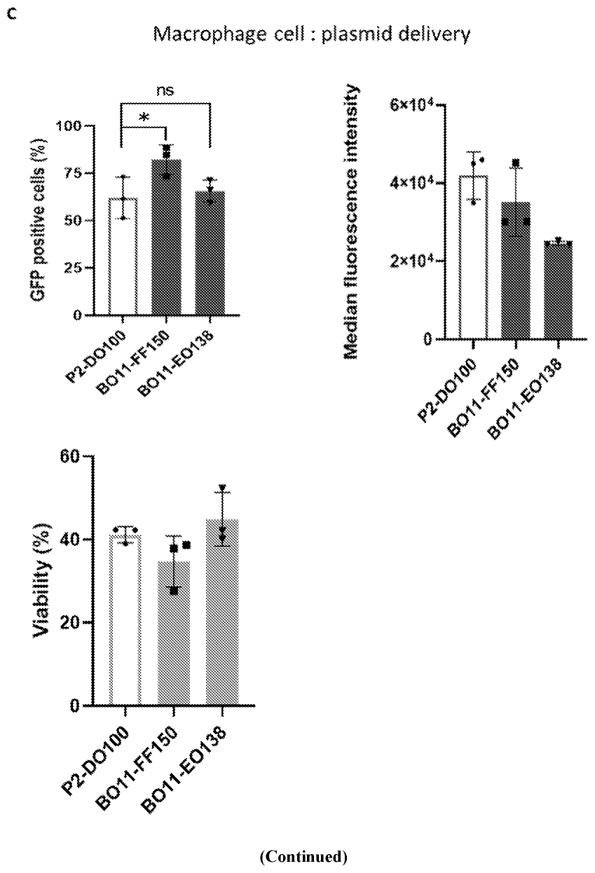

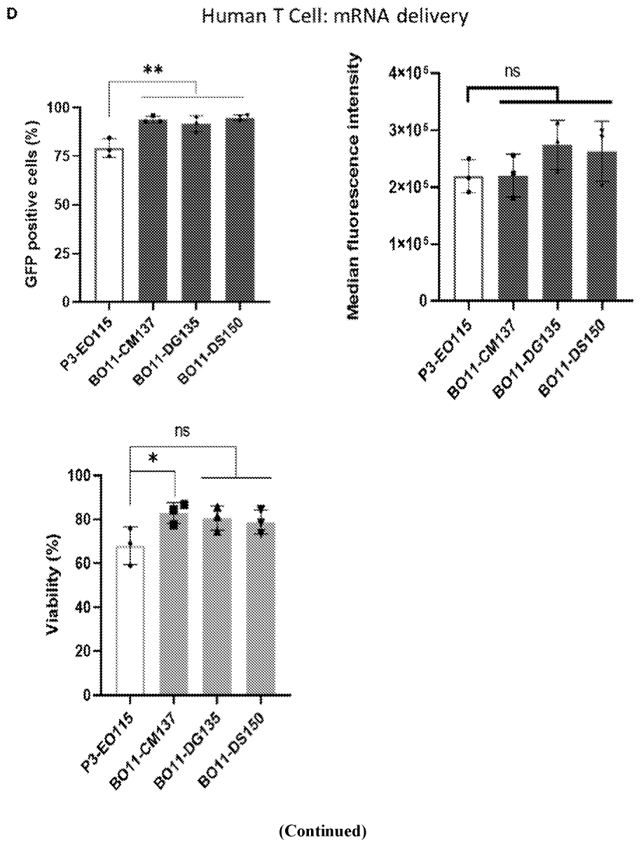

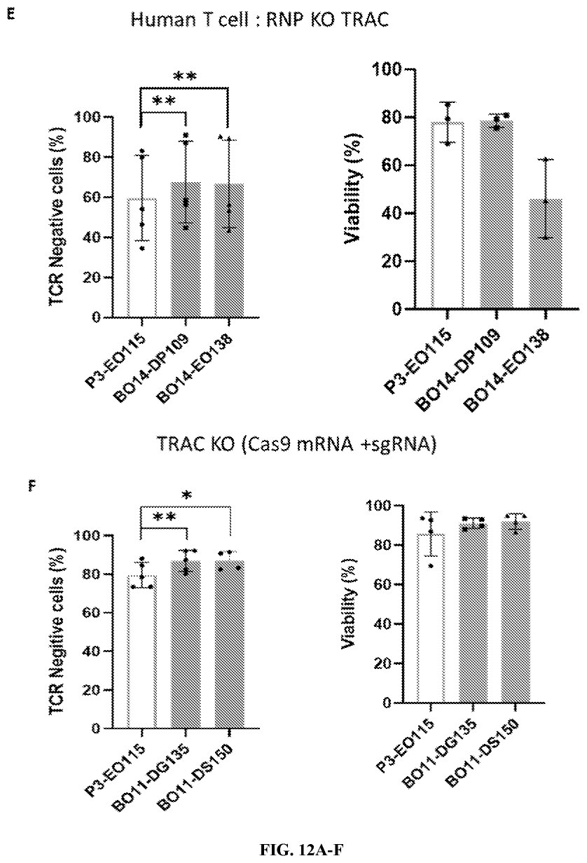

In another experiment, the EP protocol (both BO11 and BO14) was used to deliver RNP into mouse primary T and macrophage cells. As shown in FIG. 12A-F, the results were also superior.

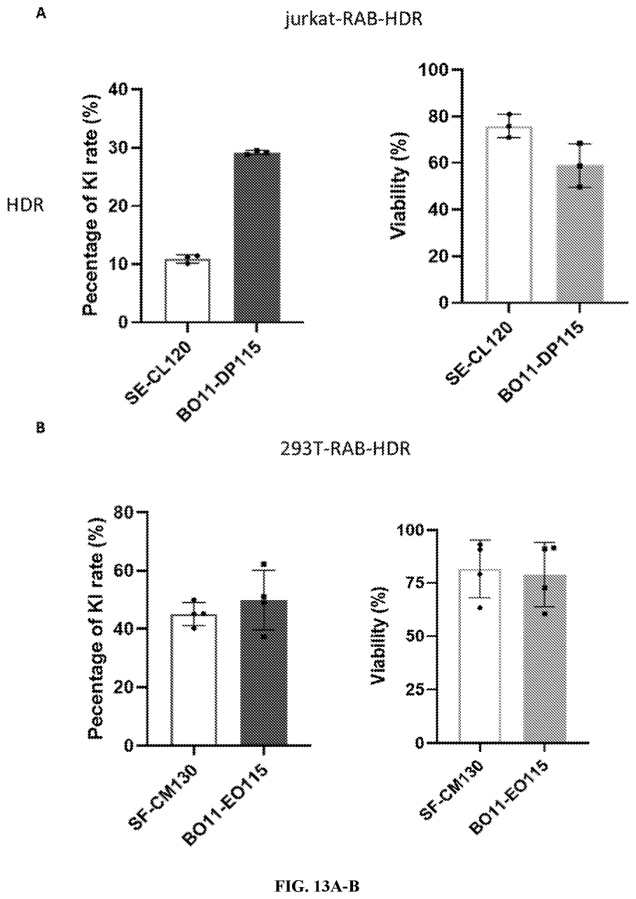

In addition, a testing was conducted for delivery of DNA for mediating Cas9-based HDR in Jurkat cells. As shown FIG. 13A-B, the BP11 buffer worked exceptionally better than the commercial buffers.

### Example 3: mRNA Delivery to Human Hematopoietic Stem and Progenitor Cells (HSPCs)

This example tested the use of the developed buffer for enhanced mRNA delivery into the human hematopoietic stem and progenitor cells (HSPC).

The buffer BO11 (1 part of buffer B and 1 part of Opti-mem) was used in this example. BO11 achieved three-fold increase in delivering mRNA to the nuclei of HSPC, when compared with the commercial standard (P3-Lonza).

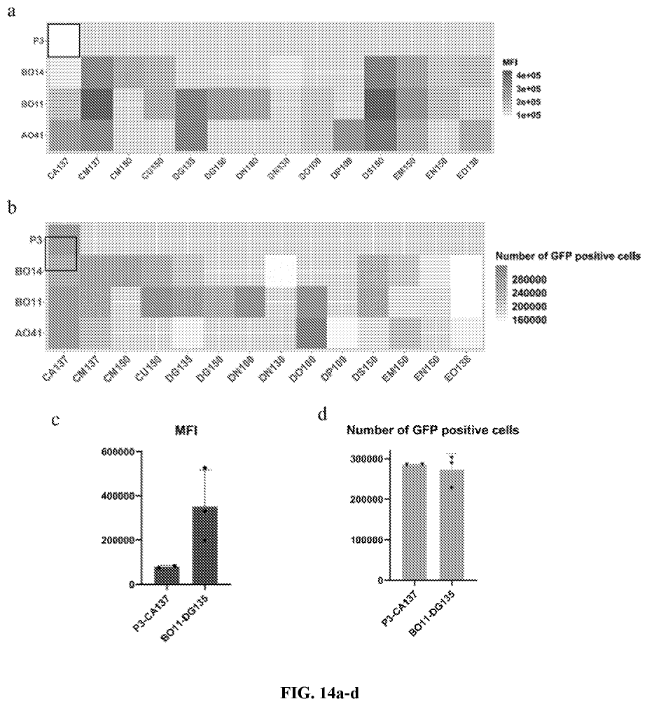

The conditions for electroporation (EP) process for BO11, BO14, A041 and P3 were optimized using GFP mRNA in human CD34+ hematopoietic stem and progenitor cells (HSPCs) (FIG. 14a-b). Analysis of median fluorescence intensity (MFI) and number of GFP positive cells 12 h post-EP by flow cytometry. When compared (FIG. 14c-d), BO11 with the DG135 electroporation program showed three-fold increase in fluorescence intensity (c) and comparable transfected cells (d) in human HSPCs as compared to P3.

### Example 4: Effects of Osmotic Pressure on mRNA Delivery

This example discovered that while an isotonic osmotic pressure (about 300 mOsmol/kg) of the electroporation buffers was optimal for dsDNA delivery efficiency, a minimum of 330 mOsmol/kg osmotic pressure was required for efficient delivery of RNA.

First, the impacts of osmotic pressure of the electroporation buffer on delivery of 0.5 μg of dsDNA (encoding GFP) to primary mouse T cells were tested. The buffers tested had osmotic pressures ranging from 190 to 512 mOsmol/kg. As shown in FIG. 16, as osmotic pressure increased, the transfection efficiency (FIG. 16B) and the fluorescence intensity (FIG. 16C) dropped, but the viability increased (FIG. 16D). Only at about the isotonic osmotic pressure (around 300 mOsmol/kg), did the highest GFP positive cells result (FIG. 16A).

Second, electroporation buffers of a range of osmotic pressures (158-500 mOsmol/kg) were tested to deliver 0.5 μg of GFP-encoding mRNA via electroporation to Jurkat cells. When the osmolality of electroporation buffer was above 330 mOsmol/kg, significantly enhanced MFI (FIG. 17A) and slightly increased viability (FIG. 17A) were observed. These data, therefore, suggest that hyper-osmolality promotes mRNA delivery.

This experiment was repeated to deliver 0.5 μg GFP-encoding mRNA to primary human T cells. Similar results were observed. When the osmolality of the electroporation buffers was above 330 mOsmol/kg, significantly enhanced GFP positive cells (FIG. 18A) and MFI (FIG. 18B) resulted, with slightly increased cell viability (FIG. 18C).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. An aqueous solution, comprising succinate, mannitol, a sugar selected from the group consisting of glucose, sucrose and inositol, glutamine or an analog thereof, and an antioxidant.

2. The aqueous solution of claim 1, wherein the sugar is D-glucose.

3. The aqueous solution of claim 1, wherein the glutamine or analog thereof is selected from the group consisting of L-alanyl-L-glutamine, L-glutamine, and D-glutamine.

4. The aqueous solution of claim 1, wherein the succinate is sodium succinate, potassium succinate, or magnesium succinate.

5. The aqueous solution of claim 1, wherein the antioxidant is sodium pyruvate or acetylcysteine.

6. The aqueous solution of claim 1, further comprising Na₂HPO₄ and NaH₂PO₄.

7. The aqueous solution of claim 1, further comprising serum.

8. The aqueous solution of claim 1, which includes less than 80 mM NaCl, or preferably less than 70 mM, 60 mM, or 50 mM NaCl.

9. The aqueous solution of claim 8, which comprises 5 mM to 30 mM sodium succinate, 1 mM to 30 mM mannitol, 3 mM to 30 mM glucose, 50 mg/L to 900 mg/L L-Glutamine or L-alanyl-L-glutamine, 0.1 mM to 0.6 mM sodium pyruvate, and includes less than 60 mM NaCl.

10. A method for introducing a polynucleotide into a cell, comprising applying a pulse of electricity to a sample comprising the polynucleotide and the cell in an aqueous solution of claim 1.

11. A method for introducing a polynucleotide into a cell, comprising applying a pulse of electricity to a sample comprising the polynucleotide and the cell in a culture medium that includes less than 80 mM NaCl, or preferably less than 70 mM, 60 mM, or 50 mM NaCl.

12. The method of claim 11, wherein the culture medium comprises succinate, mannitol, a sugar, glutamine or an analog thereof, and an antioxidant.

13. The method of claim 11, wherein the polynucleotide is DNA or RNA.

14. The method of claim 13, wherein the DNA is single-stranded DNA or double-stranded DNA.

15. The method of claim 13, wherein the RNA is siRNA, sgRNA, messenger RNA or double-stranded RNA or antisense RNA or tRNA or RNA aptamer.

16. The method of claim 15, wherein the RNA is provided in an RNA-protein complex.

17. The method of claim 11, wherein the cell is a mammalian cell.

18. The method of claim 17, wherein the culture medium further comprises an agent that inhibits the cGAS-STING pathway.

19. The method of claim 18, wherein the agent is an siRNA or antibody targeting the cGAS or STING protein.

20. (canceled)

21. A method for introducing a biomolecule into a cell, comprising applying a pulse of electricity to a sample comprising the biomolecule and the cell in an aqueous solution having an osmotic pressure greater than 310 mOsmol/kg, wherein the biomolecule is an RNA molecule, a protein, or the combination thereof.

22-36. (canceled)
